(19) United States
(12) Patent Application Publication
(10) Pub. No.: US 2025/0207155 A1
(43) Pub. Date: Jun. 26, 2025

(54) PARTICLE-BASED BIOSEPARATION ASSAYS

(71) Applicant: Seer, Inc., Redwood City, CA (US)

(72) Inventors: Robert S LANGER, Newton, MA (US); Xiaoyan ZHAO, Foster City, CA (US); Craig STOLARCZYK, Springfield, OR (US)

(21) Appl. No.: 18/395,124

(22) Filed: Dec. 22, 2023

(51) Int. Cl. C12P 1/00

(52) U.S. Cl. CPC C12P 1/00

(57) ABSTRACT

Apparatus, systems and methods for separation of biomolecules in a biofluid. Separation methods include nanoparticles-based separation (e.g., formation of biomolecule coronas on nanoparticles), serial enrichment, electrophoretic particle separation, chromatography, affinity tag, isocratic elution, gradient and isocratic elution, cleave particle coating, or any combination thereof.

### BACKGROUND

Bioseparation refers to the process of purifying biological products, often on a large-scale, using fundamental aspects of engineering and scientific principles, based on one or more of the following characteristics: density, diffusivity, electrostatic charge, polarity, shape, size, solubility and volatility. The goal of bioseparation is to refine molecules (including biomolecules), cells, or parts of cells into purified fractions.

### SUMMARY

Disclosed herein are apparatus, systems and methods for fractionating, enriching, purifying, or detecting biomolecules in a fluid.

In one aspect, provided herein is a method for fractionating a biological sample comprising a plurality of biomolecules, the method comprising: a) contacting the biological sample with a plurality of particles, thereby adsorbing the plurality of biomolecules to surfaces of the plurality of particles to form at least one biomolecule corona on the plurality of particles; b) separating at least one particle comprising a biomolecule corona from the plurality of particles to form a first fraction, wherein separating of the first fraction is based on a physicochemical property of the first fraction, wherein the at least one particle comprising the biomolecule corona in the first fraction comprises a first subset of the plurality of biomolecules of the biological sample; c) desorbing the first subset of the plurality of biomolecules of the biological sample in the first portion; d) separating at least one particle comprising a biomolecule corona from the remaining plurality of particles in step (b) to form a second fraction, wherein separating of the second fraction is based on a physicochemical property of the second fraction, wherein the at least one particle comprising the biomolecule corona in the second fraction comprises a second subset of the plurality of biomolecules of the biological sample; e) desorbing the second subset of the plurality of biomolecules of the biological sample in the second portion; and f) collecting the desorbed first and second subsets of the plurality of biomolecules, thereby fractionating the biological sample.

In some embodiments, the plurality of particles comprises at least two subsets of particles, each subset of particles differing by at least one physicochemical property.

In some embodiments, the physicochemical property is selected from the group consisting of composition, size, surface charge, hydrophobicity, hydrophilicity, roughness, density surface shape, zeta potential, and any combination thereof.

In some embodiments, the adsorption of the plurality of biomolecules to the surface of the plurality of particles results in a change of a physicochemical property.

In some embodiments, the adsorption results in a change in zeta potentials of the particles.

In some embodiments, the adsorption of biomolecules increases the zeta potentials of the particles.

In some embodiments, the adsorption of biomolecules decreases the zeta potentials of the particles.

In some embodiments, the method comprises contacting the biological sample with a plurality of particles, thereby adsorbing the plurality of biomolecules comprising at least 3, at least 4, at least 5, at least 10, at least 20, at least 35, at least 50, at least 70, or at least 90 different protein groups to surfaces of the plurality of particles.

In some embodiments, the method comprises contacting the biological sample with a plurality of particles, thereby adsorbing the plurality of biomolecules comprising at least 100, at least 150, at least 200, at least 250, at least 300, at least 400, at least 500, at least 750, or at least 900 different protein groups to surfaces of the plurality of particles.

In some embodiments, the method comprises contacting the biological sample with a plurality of particles, thereby adsorbing the plurality of biomolecules comprising at least 1000, 1500, at least 2000, at least 5000, at least 10000, at least 20000, at least 20000, at least 50000, or at least 100000 different protein groups to surfaces of the plurality of particles.

In some embodiments, desorbing the subset of the plurality of biomolecules of the biological sample from the particle comprises treating the particle with an enzyme selected from the group consisting of trypsin, chymotrypsin, endoproteinase Glu C, endoproteinase Lys C, elastase, subtilisin, proteinase K, thrombin, factor X, endoproteinase Arg C, papain, endoproteinase AspN, thermolysin, pepsin, aspartyl protease, cathepsin D, zinc mealloprotease, glycoprotein endopeptidase, aminopeptidase, prenyl protease, caspase, kex2 endoprotease, or any combination thereof.

In another aspect, provided herein is a method for fractionating one or more portions of a biological sample, comprising: a) contacting the one or more portions of a biological sample with one or more particles to allow a plurality of biomolecules from the one or more portions of a biological sample to adsorb to the one or more particle panels to form at least one biomolecule corona on the one or more particles; b) contacting the one or more particles with a first solution, thereby desorbing a first subset of biomolecules from among the plurality of biomolecules from the one or more particle panels into the first solution; c) collecting the first subset of biomolecules from the first solution; d) contacting the one or more particles with a second solution, thereby desorbing a second subset of biomolecules from among the plurality of biomolecules from the one or more particles into the second solution; and e) collecting the second subset of biomolecules from the second solution.

In some embodiments, the first solution is different from the second solution.

In some embodiments, the first solution comprises a higher concentration of an organic solvent than the second solution.

In some embodiments, the first solution comprises a lower concentration of an organic solvent than the second solution.

In some embodiments, the method comprises contacting the one or more particles with a third solution, thereby desorbing a third subset of biomolecules from among the plurality of biomolecules from the one or more particles into the third solution and collecting the third subset of biomolecules from the third solution.

In some embodiments, the first subset of biomolecules is different from the second subset of biomolecules and the third subset of biomolecules.

In some embodiments, contacting the one or more particles with a second solution is performed prior to contacting the one or more particles with a third solution and after contacting the one or more particles with a first solution.

In some embodiments, the third solution is different from the first or second solution.

In some embodiments, the one or more particles comprise at least two particles, and the at least two particles comprise a particle panel.

Additional aspects and advantages of the present disclosure may become readily apparent to those skilled in this art from the following detailed description, wherein only illustrative embodiments of the present disclosure are shown and described. As may be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

The patent or application file contains at least one drawing executed in color. Copies of this patent or patent application publication with color drawing(s) will be provided by the Office upon request and payment of the necessary fee.

FIG. 1 shows a computer system that is programmed or otherwise configured to implement methods provided herein.

FIG. 2 provides an example workflow for collecting biomolecules from a biological sample onto particles.

FIG. 3 provides an example workflow for a particle-based assay for analyzing biomolecules from a biological sample.

FIG. 4 provides an example workflow for assaying biomolecules from a biological sample with magnetic particles.

FIG. 5 illustrates the number of proteins collected on and subsequently identified by mass spectrometry following collection on particle panels comprising from 1 to 12 particles.

FIG. 6A shows a flowchart for separating biomolecules in a biological sample by separating particles.

FIG. 6B shows an example of separating particles using a particle panel.

FIG. 7A shows a flowchart for separating particles using coated nanoparticles.

FIG. 7B shows an example of biomolecule coronas forming on a coated particle.

FIG. 7C shows a flowchart for separating biomolecules using particles and serially using different elution conditions.

FIG. 8 shows a flowchart for separating biomolecules using particle panels and serially using different elution conditions.

FIG. 9 shows an example of separating biomolecules using isocratic elution.

FIG. 10 illustrates a method of separating biomolecules using a particle affinity tag attached to a particle.

FIG. 11 illustrates a method of high-throughput separation of biomolecules.

FIG. 12 shows an exemplary process of purifying a biomolecule from a depleted sample to produce a purified sample.

FIG. 13 show a flowchart for serial enrichment of biomolecules.

FIG. 14 shows an example electrophoretic separation of particles and biomolecules.

### DETAILED DESCRIPTION

Biological samples are often complex mixtures comprising vast arrays of biomolecules with disparate properties. The present disclosure provides a range of methods for fractionating, collecting, enriching or purifying biomolecules from biological samples, thereby enabling deep analysis, profiling, and biomolecule detection. In some cases, the methods comprise contacting the biomolecules in the biological sample with a plurality of particles, thereby adsorbing the biomolecules to the plurality of particles, separating biomolecules based on a physiochemical property of the particles, and desorbing a subset of biomolecules of the biological sample from the particles, thereby fractionating the biological sample.

Some aspects of the present disclosure provide compositions, systems, and methods for collecting biomolecules on particles, and separating the biomolecules collected on the particles into fractions. The fractions may be separated based on a physicochemical property of the particles and/or biomolecules.

In some embodiments, particles used for separating biomolecules in a biological sample may include one or more particles based on a physicochemical property. In some instances, a particle panel comprising particles of different physicochemical properties may be used. A particle panel disclosed herein can be used in methods of corona analysis to detect thousands of proteins across a wide dynamic range. In some embodiments, the particle panel can be used in methods of corona analysis to detect thousands of proteins across a wide dynamic range in the span of hours. In some embodiments, the particle panel can be used in methods of corona analysis to detect thousands of proteins across a wide dynamic range in less than ten hours. In some embodiments, the particle panel can be used in methods of corona analysis to detect thousands of proteins across a wide dynamic range in less than five hours. In some embodiments, the particle panel can be used in methods of corona analysis to detect thousands of proteins across a wide dynamic range in less than three hours. In some embodiments, the particle panel can be used in methods of corona analysis to detect thousands of proteins across a wide dynamic range in less than one hour. In some embodiments, the particle panel can be used in methods of corona analysis to detect thousands of proteins across a wide dynamic range in less than thirty minutes.

A method of the present disclosure may comprise contacting a biological sample (e.g., plasma) with a particle under conditions suitable for biomolecule collection (e.g., non-covalent adsorption) on the particle. The collection of biomolecules on the surface of the particle may be referred to as a ‘biomolecule corona’. The biomolecule corona that forms on a particle may comprise a complex mixture of biomolecules from the biological sample. The biomolecule corona may compress the abundance ratios of biomolecules from a sample, thereby enabling analysis of dilute, and in many cases difficult to analyze, biomolecules.

Biomolecules collected on particles may be subjected to further analysis. In some cases, a method may comprise collecting a biomolecule corona or a subset of biomolecules from a biomolecule corona. In some cases, the collected biomolecule corona or the collected subset of biomolecules from the biomolecule corona may be subjected to further particle-based analysis (e.g., particle adsorption). In some cases, the collected biomolecule corona or the collected subset of biomolecules from the biomolecule corona may be purified or fractionated (e.g., by a chromatographic method). In some cases, the collected biomolecule corona or the collected subset of biomolecules from the biomolecule corona may be analyzed (e.g., by mass spectrometry).

A biomolecule corona may include nucleic acids, small molecules, proteins, lipids, polysaccharides, or any combination thereof, adsorbed to the surface of a particle form a sample in which the particle is incubated, nucleic acid, a small molecule, a protein, a lipid, a polysaccharide, or any combination thereof.

### Biomolecule Fractionation Methods

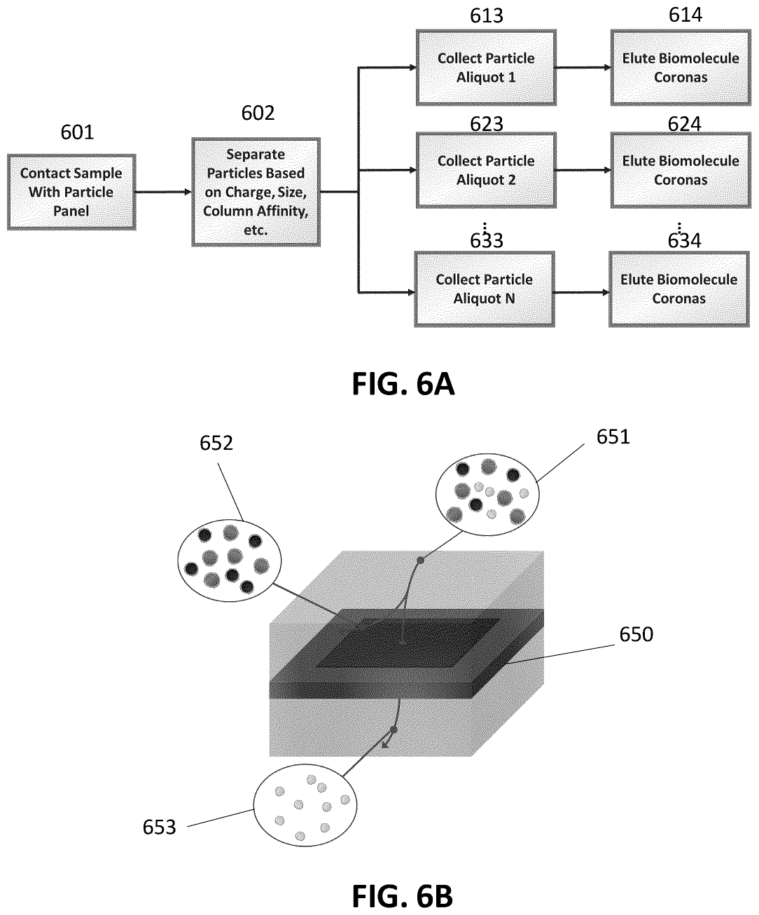

FIG. 6A illustrates an example of a method of separating components of a biological sample into different fractions. A biological sample may comprise at least 2 classes of proteins, 3 classes of proteins, 4 classes of proteins, 5 classes of proteins, 6 classes of proteins, 7 classes of proteins, classes of proteins, 8 classes of proteins, 9 classes of proteins, or 10 classes of proteins. In step 601, a biological sample is contacted with a particle panel having different types of particles, thereby adsorbing biomolecules of the biological sample to the particles. The biological sample may also, in some cases, be interrogated with a plurality of particles, the particles having the same or different physicochemical properties. In some cases, the biological sample is contacted with the particle panel multiple times (e.g., 2, 3, 4, 5, 6, 7, 8, 9, 10, 11, 12, 13, 14, 15, 16, 17, 18, 19, 20, or more times). In some cases, the particle panel has multiple types of particles, for example, 2, 3, 4, 5, 6, 7, 8, 9, 10, 11, 12, 13, 14, 15, 16, 17, 18, 19, 20, or more types of particles. It is contemplated that the above methods and compositions enable formation of highly complex protein coronas, for example, protein coronas comprising at least 100 unique proteins, at least 120 unique proteins, at least 140 unique proteins, at least 160 unique proteins, at least 180 unique proteins, at least 200 unique proteins, at least 220 unique proteins, at least 240 unique proteins, at least 260 unique proteins, at least 280 unique proteins, at least 300 unique proteins, at least 320 unique proteins, at least 340 unique proteins, at least 360 unique proteins, at least 380 unique proteins, at least 400 unique proteins, at least 420 unique proteins, at least 440 unique proteins, at least 460 unique proteins, at least 480 unique proteins, at least 500 unique proteins, at least 520 unique proteins, at least 540 unique proteins, at least 560 unique proteins, at least 580 unique proteins, at least 600 unique proteins, at least 620 unique proteins, at least 640 unique proteins, at least 660 unique proteins, at least 680 unique proteins, at least 700 unique proteins, at least 720 unique proteins, at least 740 unique proteins, at least 760 unique proteins, at least 780 unique proteins, at least 800 unique proteins, at least 820 unique proteins, at least 840 unique proteins, at least 860 unique proteins, at least 880 unique proteins, at least 900 unique proteins, at least 920 unique proteins, at least 940 unique proteins, at least 960 unique proteins, at least 980 unique proteins, at least 1000 unique proteins, at least 2000 unique proteins, at least 3000 unique proteins, at least 4000 unique proteins, at least 5000 unique proteins, at least 6000 unique proteins, at least 7000 unique proteins, at least 8000 unique proteins, at least 9000 unique proteins, at least 10000 unique protein, at least 20000 unique proteins, at least 30000 unique proteins, at least 40000 unique proteins, at least 50000 unique proteins, at least 60000 unique proteins, at least 70000 unique proteins, at least 80000 unique proteins, at least 90000 unique proteins, or at least 100000 unique protein.

In some cases, a method of the present disclosure may identify a large number of unique biomolecules (e.g., proteins) in a biological sample (e.g., a biofluid). In some cases, a surface disclosed herein may be incubated with a biological sample to adsorb at least 1000, 2000, 3000, 4000, 5000, 6000, 7000, 8000, 9000, or 10000 unique biomolecules. In some cases, a surface disclosed herein may be incubated with a biological sample to adsorb at most 1000, 2000, 3000, 4000, 5000, 6000, 7000, 8000, 9000, or 10000 unique biomolecules. In some cases, a surface disclosed herein may be incubated with a biological sample to adsorb at least 1000, 2000, 3000, 4000, 5000, 6000, 7000, 8000, 9000, or 10000 unique biomolecule groups. In some cases, a surface disclosed herein may be incubated with a biological sample to adsorb at most 1000, 2000, 3000, 4000, 5000, 6000, 7000, 8000, 9000, or 10000 unique biomolecule groups. In some cases, several different types of surfaces can be used, separately or in combination, to identify large numbers of proteins in a particular biological sample. In other words, surfaces can be multiplexed in order to bind and identify large numbers of biomolecules in a biological sample.

In some cases, the particles and particle panels disclosed herein can be used to identify a number of proteins, peptides, protein groups, or protein classes using a protein analysis workflow described herein (e.g., a protein corona analysis workflow). In some cases, the particles and particle panels disclosed herein can be used to identify at least 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 unique proteins. In some cases, the particles and particle panels disclosed herein can be used to identify at most 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 unique proteins. In some cases, the particles and particle panels disclosed herein can be used to identify at least 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 protein groups. In some cases, the particles and particle panels disclosed herein can be used to identify at most 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 protein groups. In some cases, the particles and particle panels disclosed herein can be used to identify at least 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, or 1000000 peptides. In some cases, the particles and particle panels disclosed herein can be used to identify at most 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, or 1000000 peptides. In some cases, a peptide may be a tryptic peptide. In some cases, a peptide may be a semi-tryptic peptide. In some cases, protein analysis may comprise contacting a sample to distinct surface types (e.g., a particle panel), forming adsorbed biomolecule layers on the distinct surface types, and identifying the biomolecules in the adsorbed biomolecule layers (e.g., by mass spectrometry). Feature intensities, as disclosed herein, may refer to the intensity of a discrete spike (“feature”) seen on a plot of mass to charge ratio versus intensity from a mass spectrometry run of a sample. In some cases, these features can correspond to variably ionized fragments of peptides and/or proteins. In some cases, using the data analysis methods described herein, feature intensities can be sorted into protein groups. In some cases, protein groups may refer to two or more proteins that are identified by a shared peptide sequence. In some cases, a protein group can refer to one protein that is identified using a unique identifying sequence. For example, if in a sample, a peptide sequence is assayed that is shared between two proteins (Protein 1: XYZZX and Protein 2: XYZYZ), a protein group could be the “XYZ protein group” having two members (protein 1 and protein 2). In some cases, if the peptide sequence is unique to a single protein (Protein 1), a protein group could be the “ZZX” protein group having one member (Protein 1). In some cases, each protein group can be supported by more than one peptide sequence. In some cases, protein detected or identified according to the instant disclosure can refer to a distinct protein detected in the sample (e.g., distinct relative other proteins detected using mass spectrometry). In some cases, analysis of proteins present in distinct coronas corresponding to the distinct surface types in a panel yields a high number of feature intensities. In some cases, this number decreases as feature intensities are processed into distinct peptides, further decreases as distinct peptides are processed into distinct proteins, and further decreases as peptides are grouped into protein groups (two or more proteins that share a distinct peptide sequence).

In step 602, the particles are separated based on at least a physiochemical property of the particle. The physiochemical property can be size, charge, hydrophobicity, structure, column affinity, magnetic strength, roughness, density surface functionalization, surface topography, surface curvature, porosity, core material, shell material, shape, affinity, and any combination thereof.

After the particles are separated, particle aliquot 1 is collected (613), particle aliquot 2 is collected (623), and so forth, till particle aliquot N is collected (633). In steps 614, 624 and 643, biomolecule coronas are eluted from separated particle aliquots (e.g., 1, 2, 3, 4, 5, 6, 7, 8, 9, 10, 11, 12, 13, 14, 15, 16, 17, 18, 19, 20, or more portions), thereby desorbing the biomolecules from the biomolecule coronas. As used herein, “desorb” can mean to cause the release of one or more biomolecules from a surface of a particle. In some cases, the biomolecules are desorbed by using a lysis buffer comprising a. In some cases, the biomolecules are desorbed by detergents (e.g., Triton X-100 or SDS). In some cases, the biomolecules are desorbed by using one or more proteases selected from among the group consisting of trypsin, chymotrypsin, endoproteinase Glu C, endoproteinase Lys C, elastase, subtilisin, proteinase K, thrombin, factor X, endoproteinase Arg C, papaine, endoproteinase Asp N, thermolysine, pepsin, aspartyl protease, cathepsin D, zinc mealloprotease, glycoprotein endopeptidase, aminopeptidase, prenyl protease, caspase, kex2 endoprotease, or any combination thereof. In some cases, the particles are heated (e.g., 37° C.) and agitated during the desorbing process. It is contemplated that the particle panel may adsorb proteins of high, medium, and low abundance in the biological sample. For example, particle aliquot 1 may be enriched in high abundance proteins, and particle aliquot 2 may be enriched in low abundance proteins. Separation of the biological sample with various particle types enables detection of a low abundance proteins in the biological sample. Biomolecules separated using particles may be further purified or fractionated and analyzed (e.g., by mass spectrometry). In some cases, further purification and fractionation may be performed using chromatography, gel electrophoresis, ion-exchange chromatography, affinity chromatography, size exclusion chromatography, or reverse phase chromatography.

FIG. 6B is an example of separating a biological sample containing at least three protein classes 651 is contacted with a particle panel 650, and separated into two fractions 652 and 653. In this example, fraction 653 only has one class of protein, while fraction 653 has two protein classes.

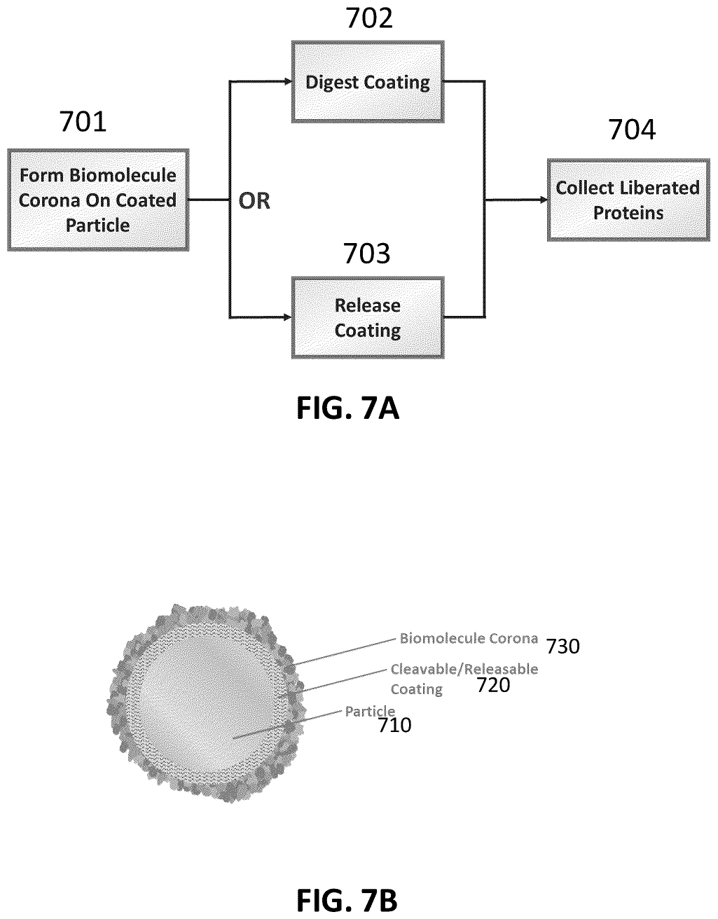

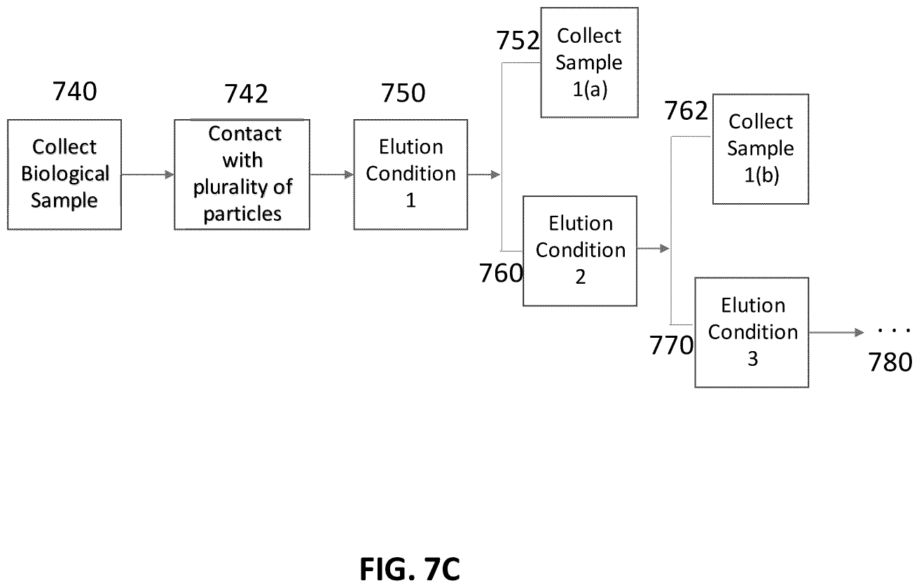

FIG. 7A shows an example of a method of separating biomolecules from a biological sample using a particle with cleavable or releasable coating. In Step 701, a coated particle is contacted with a biological sample, thereby forming one or more biomolecule coronas on the coated particle. The coating of the particle is digested (Step 702) or released (Step 703), thereby releasing the coating and biomolecule coronas from the particle. The liberated proteins are collected Step 704.

In FIG. 7B, a particle 710 is coated with a cleavable or releasable coating 720. After the coated particle is contacted with a biological sample, biomolecule coronas 730 form on the cleavable or releasable coating 720 of the particle 710. In some instances, the cleavable or releasable coating 720 is a nucleic acid (e.g., DNA or RNA), which can be cleaved or released by treating with an enzyme (e.g., deoxyribonuclease for DNA, or ribonuclease for RNA). In some instances, the cleavable or releasable coating 720 is a lipid, which can be cleaved or released by treating with a lipase and lipoxygenase. In some instances, the cleavable or releasable coating 720 is a protein, which can be cleaved or released by treating with a protease. In some instances, the cleavable or releasable coating 720 is a protein, which can be cleaved or released by treating with a protease. In some instances, the cleavable or releasable coating 720 is a metal that reacts with an acid (e.g., zinc or iron), which can be cleaved or released by treating with an acid (e.g., hydrochloric acid, or sulfuric acid). In some instances, the cleavable or releasable coating 720 is a resin, which can be released by heating. In some instances, the cleavable or releasable coating 720 is a degradable polymer. In some instances, the methods of releasing the cleavable or releasable coating may be chemical release of the coating, enzymatic release of the coating, heat induced release of the coating, photo-induced release of the coating, release of the coating by shifting the equilibrium (e.g., through competitive elution), repeated heating and freezing the coated particles, grinding the coated particles, or subjecting the coated particles to ultrasound, or any combination thereof.

FIG. 7C shows a flowchart for a method for separating biomolecules using particles and serially using different elution conditions. In step 740, a biological sample comprising a plurality of biomolecules is collected. In step 742, the biological sample comprising a plurality of biomolecules is contacted with a plurality of particles, thereby adsorbing the plurality of biomolecules to surfaces of the plurality of particles to form at least one biomolecule corona on the plurality of particles. In step 750, the plurality of particles with at least one biomolecule corona is eluted under Elution Condition 1, in which a first subset of the plurality of biomolecules of the biological sample in a first fraction is desorbed and collected as Sample 1(a) 752. In step 760, the plurality of particles with at least one biomolecule corona is eluted under Elution Condition 2, in which a second subset of the plurality of biomolecules of the biological sample in a second portion is desorbed and collected as Sample 1(b) 762. In step 770, the plurality of particles with at least one biomolecule corona is eluted under Elution Condition 3, in which a third subset of the plurality of biomolecules of the biological sample in a third portion is desorbed, and in step 780 the elution processes previously described can be performed repeatedly (i.e., greater than three elution condition steps) to collect additional samples. In some cases, the method provided herein comprises separating at least one particle comprising a biomolecule corona from the plurality of particles to form a first fraction, wherein separating of the first fraction is based on a physicochemical property of the first fraction, wherein the at least one particle comprising the biomolecule corona in the first fraction comprises a first subset of the plurality of biomolecules of the biological sample. In some cases, the term “elution” refers to the process of desorbing biomolecule coronas from particles. In some cases, the method provided herein comprises separating at least one particle comprising a biomolecule corona from the plurality of particles to form a second fraction, wherein separating of the second fraction is based on a physicochemical property of the second fraction, wherein the at least one particle comprising the biomolecule corona in the second fraction comprises a second subset of the plurality of biomolecules of the biological sample. In some cases, the plurality of particles comprises at least two subsets of particles, each subset of particles differing by at least one physicochemical property. In some cases, the physicochemical property is selected from the group consisting of composition, size, surface charge, hydrophobicity, hydrophilicity, roughness, density surface functionalization, surface topography, surface curvature, porosity, core material, shell material, shape, zeta potential, and any combination thereof. In some cases, the adsorption of the plurality of biomolecules to the surface of the plurality of particles results in a change of a physicochemical property. In some cases, the adsorption results in a change in zeta potentials of the particles. In some cases, the adsorption of biomolecules increases the zeta potentials of the particles. In some cases, the adsorption of biomolecules decreases the zeta potentials of the particles. In some cases, the method provided herein comprises contacting the biological sample with a plurality of particles, thereby adsorbing the plurality of biomolecules comprising at least 3, at least 4, at least 5, at least 10, at least 20, at least 35, at least 50, at least 70, or at least 90 different protein groups to surfaces of the plurality of particles. In some cases, the method provided herein comprises contacting the biological sample with a plurality of particles, thereby adsorbing the plurality of biomolecules comprising at least 100, at least 150, at least 200, at least 250, at least 300, at least 400, at least 500, at least 750, or at least 900 different protein groups to surfaces of the plurality of particles. In some cases, the method provided herein comprises contacting the biological sample with a plurality of particles, thereby adsorbing the plurality of biomolecules comprising at least 1000, 1500, at least 2000, at least 5000, at least 10000, at least 20000, at least 20000, at least 50000, or at least 100000 different protein groups to surfaces of the plurality of particles. In some cases, desorbing the subset of the plurality of biomolecules of the biological sample from the particle comprises treating the particle with an enzyme selected from the group consisting of trypsin, chymotrypsin, endoproteinase Glu C, endoproteinase Lys C, elastase, subtilisin, proteinase K, thrombin, factor X, endoproteinase Arg C, papain, endoproteinase AspN, thermolysin, pepsin, aspartyl protease, cathepsin D, zinc mealloprotease, glycoprotein endopeptidase, aminopeptidase, prenyl protease, caspase, kex2 endoprotease, or any combination thereof.

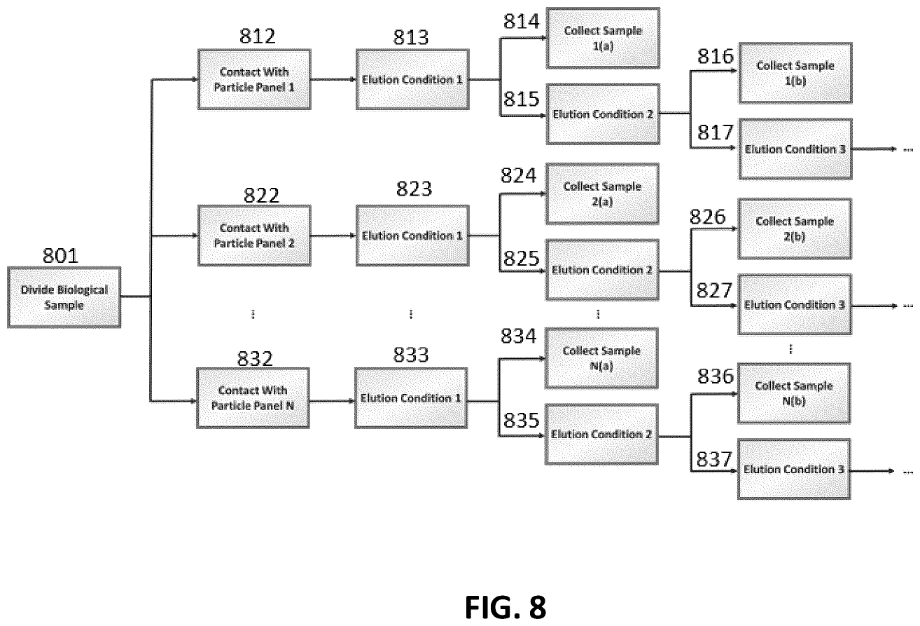

Gradient Elution. The present disclosure also provides methods of fractionating a biological sample using gradient elution, as illustrated in FIG. 8. In Step 801, a biological sample is divided into a desired portions (e.g., 1, 2, 3, 4, 5, 6, 7, 8, 9, 10, 11, 12, 13, 14, 15, 16, 17, 18, 19, 20, or more portions) prior to contacting with a particle panel. In Step 812, a first portion of the biological sample is contacted with Particle Panel 1. In Step 813, the Particle Panel 1 is eluted using a first solution under Elution Condition 1. In some cases, as used herein, the term “elution” refers to the process of desorbing biomolecule coronas from particles by washing the particles with a solution. In some cases, the first solution comprises water with one or more organic solvents (e.g., acetonitrile, methanol, alcohol, tetrahydrofuran, or isopropanol). In some cases, the first solution is water-free, comprising only one or more organic solvents. In some cases, the first solution contains acids (e.g., formic, phosphoric or trifluoroacetic acid) or salts. Contacting the Particle Panel 1 with the first solution results in desorbing a first subset of biomolecules from Particle Panel 1, which is then collected as sample 1(a) (Step 814). In Step 815, Particle Panel 1 (after being contacted with the first solution) is then contacted with a second solution, under Elution Condition 2, thereby desorbing a second subset of biomolecules from Particle Panel 1, which is collected as Sample 1(b) (Step 816). In some cases, the second solution is different from the first solution, and Elution Condition 2 is different from Elution Condition 1. In some cases, the second solution has a higher concentration of an organic solvent than the first solution. It is contemplated that the first subset of biomolecules are different from the second subset of biomolecules, and Sample 1(b) is different from Sample 1(a). In Step 817, Particle Panel 1 (after being contacted with the second solution) is then contacted with a third solution, under Elution Condition 3, thereby desorbing a third subset of biomolecules from the particle panel, which is then collected as Sample 1(c). In some cases, the third solution is different from the first or second solution, and Elution Condition 3 is different from Elution Condition 1 or 2. In some cases, the third solution has a higher concentration of an organic solvent than the second solution. It is contemplated that the third subset of biomolecules are different from the first or second subset of biomolecules, and Sample 1(c) is different from Sample 1(a) or Sample 1(b).

In Step 822, a second portion of the biological sample is contacted with Particle Panel 2. Particle Panel 2 is different from Particle Panel 1 in at least one aspect, for example, comprising different types or number of particles. In Step 823, Particle Panel 2 is eluted using a first solution under Elution Condition 1. Contacting the Particle Panel 2 with the first solution results in desorbing a first subset of biomolecules from Particle Panel 2, which is then collected as sample 2(a) (Step 824). In Step 825, Particle Panel 2 is then contacted with a second solution, under Elution Condition 2, thereby desorbing a second subset of biomolecules from the particle panel, which is collected as Sample 2(b) (Step 826). In Step 827, Particle Panel 2 is then contacted with a third solution, under Elution Condition 3, thereby desorbing a third subset of biomolecules from Particle. Panel 2, which is then collected as Sample 2(c). It is contemplated that the Sample 2(a) is different from Sample 1(a), since Particle Panel 2 is different from Particle Panel 1. Similarly, Sample 2(b) is different from Sample 1(b).

The same or similar steps 812-817 or 822-827 may be repeated for any desired times (e.g., 1, 2, 3, 4, 5, 6, 7, 8, 9, 10, 11, 12, 13, 14, 15, 16, 17, 18, 19, 20, or more portions) using different particle panels, for example, for Particle Panel N (832-837).

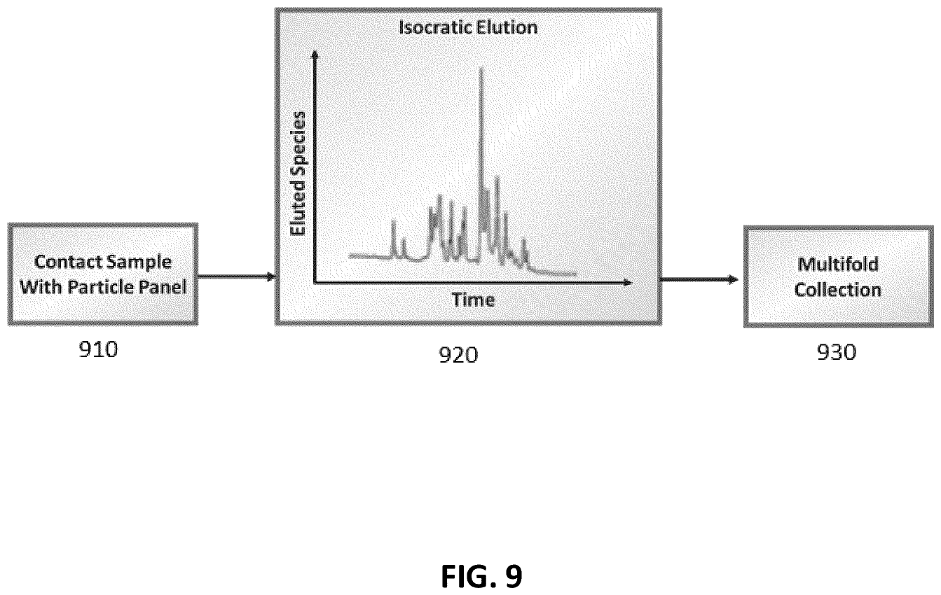

FIG. 9 is an example of separating and enriching biomolecules using isocratic elution. As used herein, the term “isocratic elution” means desorbing biomolecule coronas from particles by washing the particles with a solution having a constant concentration throughout the process. In some cases, the solution comprises water with one or more organic solvents (e.g., acetonitrile, methanol, alcohol, tetrahydrofuran, or isopropanol). In some cases, the solution is water-free, comprising only one or more organic solvents. In some cases, the solution contains acids (e.g., formic, phosphoric or trifluoroacetic acid) or salts.

In Step 910, a biological sample is contacted with a particle panel to allow biomolecules from the biological sample to adsorb to the surfaces of the particles. In Step 920, the particle panel is then eluded with a fluid to desorb a first subset of the plurality of biomolecules from the particle panel at a first time, a second subset of the plurality of biomolecules from the particle panel at a second time, and so forth, until all biomolecules are desorbed from the particle panel. In some cases, the second time and the first time is at least at least 1 second part, 2 seconds apart, 3 seconds apart, 4 seconds apart, 5 seconds apart, 5 seconds apart, 6 seconds apart, 7 seconds apart, 8 seconds apart, 9 seconds apart, 10 seconds apart, 20 seconds apart, 30 seconds apart, 45 seconds apart, 1 minute apart, 2 minutes apart, 3 minutes apart, 4 minutes apart, 5 minutes apart, 5 minutes apart, 6 minutes apart, 7 minutes apart, 8 minutes apart, 9 minutes apart, 10 minutes apart, 15 minutes apart, 20 minutes apart, 25 minutes apart, 30 minutes apart, 45 minutes apart, 60 minutes apart, 1.5 hours apart, 2 hours apart, 3 hours apart, 4 hours apart, 5 hours apart, 5 hours apart, 6 hours apart, 7 hours apart, 8 hours apart, 9 hours apart, 10 hours apart, 15 hours apart, 20 hours apart, 25 hours apart, 30 hours apart, 45 hours apart, or 60 hours apart. As shown on the graph, different biomolecules are desorbed at different rates. Different species are eluted at different time points with different concentrations are collected in Step 930. For example, an elution is collected at a peak of the graph in 920 would be enriched with a particular biomolecule, compared with the starting biological sample.

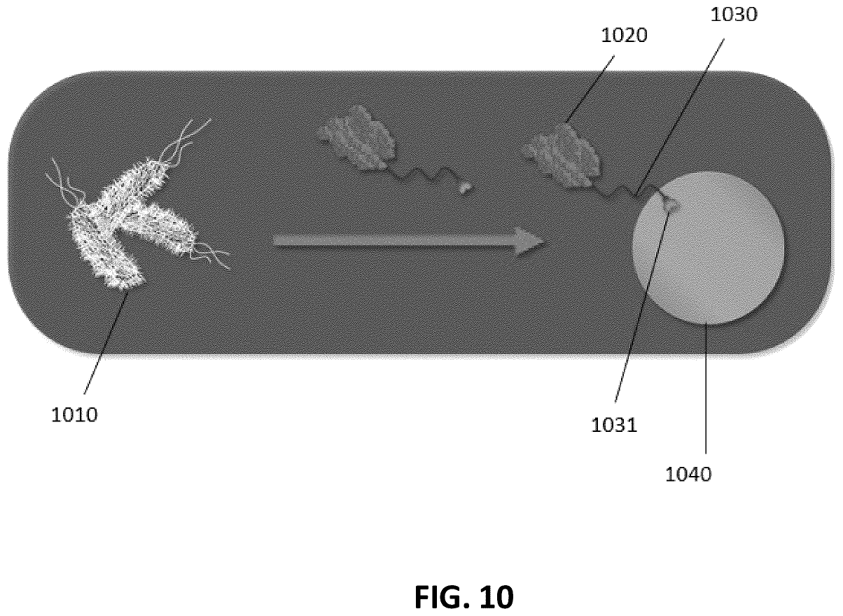

Purification of Biomolecule using Particle Affinity Tag. FIG. 10 illustrates a method of purifying a biomolecule 1020 using a particle affinity tag 1030. In this example, the biomolecule 1020 is expressed in a bacterial cell 1010, wherein the biomolecule 1020 is coupled to a particle affinity tag 1030. It is contemplated other cell types can be used to express the biomolecule, including an insect cell, a plant cell, or a mammalian cell (e.g., human cell line), or an immortalized cell line. The particle affinity tag 1030 has an affinity for a surface of a particle 1040, and can be adsorbed to the surface of particle 1040, thereby coupling the biomolecule 1020 to the particle 1040. The affinity tag 1030 can be coupled to the biomolecule 1020 by any appropriate means. In some cases, the particle affinity tag 1030 can be co-expressed in the bacterial cell 1010 with the biomolecule 1020, forming a fusion protein comprising the biomolecule 1020 and the particle affinity tag 1030. In some cases, the particle affinity tag 1030 is a made separately and added to the biomolecule 1020 produced in the bacterial cell 1010, so that the particle affinity tag 1030 is coupled to the biomolecule 1020 outside the bacterial cell 1010. For example, the particle affinity tag 1030 can be a peptide that, on one end, has an affinity for the biomolecule 1020, and on the other end, has an affinity for the particle 1040. In some cases, the particle affinity tag 1030 comprises a first end 1031 that binds to the surface of the particle 1040. In some instances, the first end 1031 comprises a peptide or domain that has affinity to the surface of the particle 1040. For example, the shape of first end 1031 is complementary to at least a portion of the surface of the particle 1040. In some cases, the particle affinity tag 1030 can be dissociated from the biomolecule 1020, thereby releasing the biomolecule 1020 from the particle 1040. In some cases, the particle 1040 is subject to a chemical condition to lower the affinity of the particle affinity tag 1030 for the particle 1020. In some instances, the methods of dissociating the affinity tag may be chemical dissociation, enzymatic dissociation, heat-induced dissociation, photo-induced dissociation, dissociation by shifting the equilibrium (e.g., through competitive elution), repeated heating and freezing the coated particles, grinding the coated particles, or subjecting the coated particles to ultrasound, or any combination thereof.

In some cases, the tag may be selected from albumin-binding protein (ABP), alkaline phosphate (AP), AU1 epitope, AU5 epitope, bacteriophase T7 epitope (T7-tag), bacteriophase V5 epitope (V5-tag), biotin-carboxy carrier protein (BCCP), bluetongue virus tag (B-tag), calmodulin binding peptide (CBP), chkloramphenicol acetyl transferase (CAT), cellulose binding domain (CBP), chitin binding domain (CBD), choline-binding domain (CBD), dihydrofolate reductase (DHFR), E2 epitope, FLAG epitope, galactose-binding protein (GBP), green fluorescent protein (GFP), glu-glu (EE-tag), glutathione S-transferase (GST), human influenza hemagglutin (HA), HaloTag®, histidine affinity tag (HAT), horseradish peroxide (HRP), HSV epitope, ketosteroid isomerase (KSI), KT3 epitope, LacZ, Luciferase, maltose-binding protein (MBP), Mye epitope, NusA, PDZ ligand, polyarginine (Arg-tag), polyaspartate (Asp-tag), polycystein (Cys-tag), polyhistidine (His-tag), polyphenylalanine (Phe-tag), profinity exact, Protein C, S1-tag, S-tag, streptavidine-binding peptide (SBP), staphylococcal protein A (Protein A), staphylococcal protein G (Protein G), Ster-tag, streptavidin, small ubiquitin-like modifier (SUMO), tandem affinity purification (TAP), T7 epitope, thioredoxin (Trx), TrpE, ubiquitin, universal, and VSV-G.

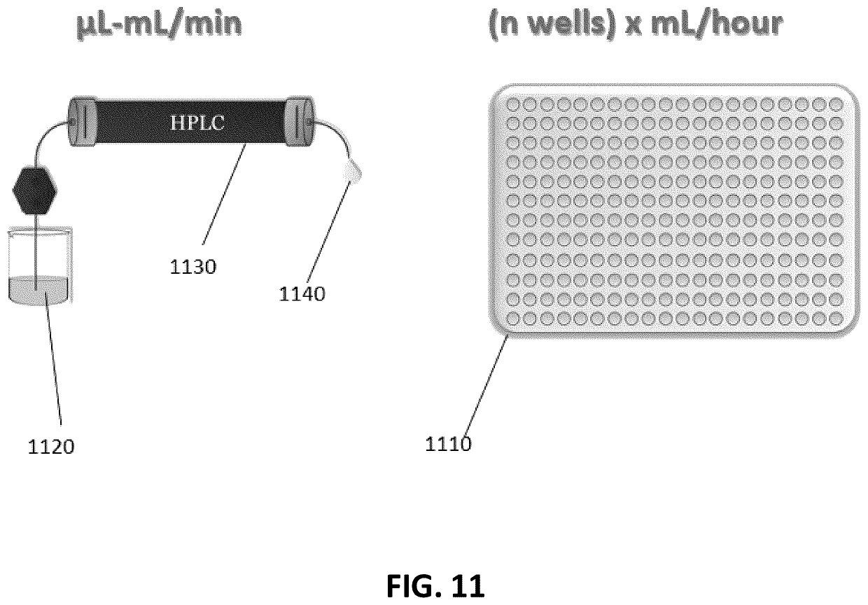

High-Throughput Separation. FIG. 11 illustrates a method of a high-throughput separation of biomolecules in a biological sample using a plurality of particle panels. In FIG. 11, a biological sample 1120 with a plurality of biomolecules is contacted with a 96-well plate comprising a plurality of particle panels 1110 to form a plurality of particle-sample mixtures. Each well on the 96-well plate is a particle panel. In some cases, the plurality of particle-sample mixtures are incubated to permit adsorption of biomolecules from the plurality of biomolecules to the surfaces of particles of the plurality of particle panels 1110. In some cases, the method further comprises collecting at least a subset of the particle-adsorbed biomolecules, thereby generating a plurality of enriched samples, wherein the subset of the particle-adsorbed biomolecules comprises at least 5% of the mass of the plurality of biomolecules, and wherein incubating and the collecting are performed in less than 90 minutes. In some cases, the biological sample 1120 is first subjected to affinity separation e.g., by reverse phase High-performance liquid chromatography (rpHPLC) to decomplex, purify or concentrate biomolecules 1130 to produce a purified sample 1140, which is then contacted with a plurality of particle panels 1110. In some cases, the incubating and the collecting may be performed in less than 50 minutes, less than 60 minutes, less than 70 minutes, less than 80 minutes, less than 90 minutes, less than 100 minutes, less than 110 minutes, less than 120 minutes, less than 130 minutes, less than 140 minutes, less than 150 minutes, not longer than 150 minutes, not longer than 140 minutes, not longer than 140 minutes, not longer than 130 minutes, not longer than 120 minutes, not longer than 110 minutes, not longer than 100 minutes, not longer than 90 minutes, not longer than 80 minutes, not longer than 70 minutes, not longer than 60 minutes, not longer than 50 minutes, between 50 minutes and 150 minutes, between 60 minutes and 140 minutes, between 70 minutes and 130 minutes, between 80 minutes and 120 minutes, between 90 minutes and 110 minutes, or between 100 minutes and 110 minutes. In some cases, the particle-adsorbed biomolecules comprise at least 1%, at least 2%, at least 3%, at least 4%, at least 5%, at least 6%, at least 7%, at least 8%, at least 9%, or at least 10% of the mass of the plurality of biomolecules. In some cases, a particle of the plurality of particles may adsorb an average of at least 100 Daltons (Da) biomolecules per nm² of its surface area. In some cases, a particle of the plurality of particles may adsorb an average of at least 50 Da, at least 60 Da, at least 70 Da, at least 80 Da, at least 90 Da, at least 100 Da, at least 110 Da, at least 120 Da, at least 130 Da, at least 140 Da, at least 150 Da biomolecules per nm² of its surface area. In some cases, a particle of the plurality of particles may adsorb an average of at least 1 protein per 15 nm² of its surface area. In some cases, a particle of the plurality of particles may adsorb an average of at least 2 proteins, at least 3 proteins, at least 4 proteins, at least 5 proteins, or at least 6 proteins per 15 nm² of its surface area.

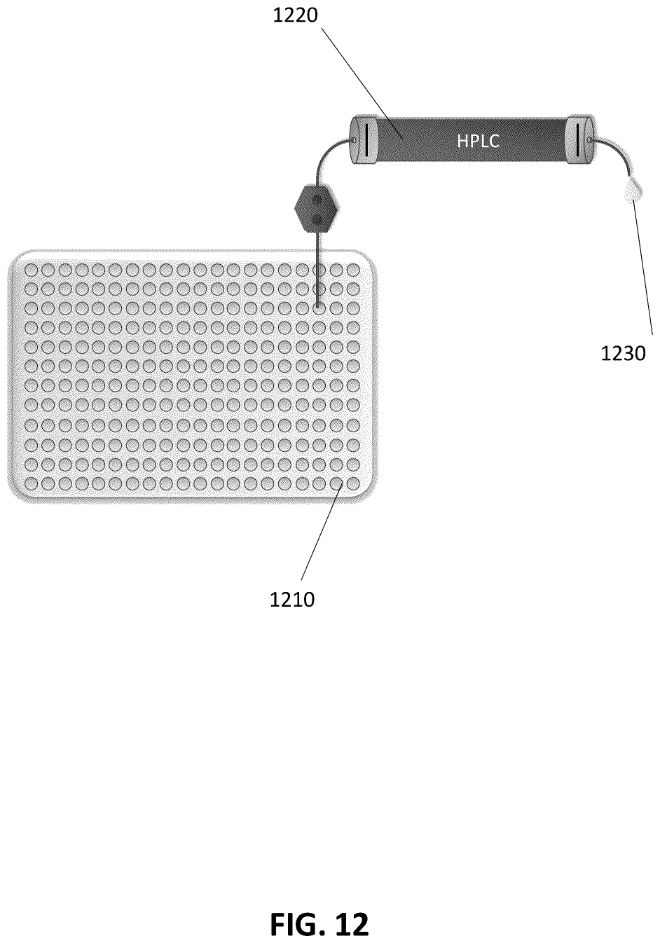

Depletion using Chromatographical Separation. In FIG. 12, a biological sample is contacted with a 96-well plate comprising a plurality of particle panels 1210, thereby forming a plurality of biomolecule coronas on the plurality of particles. The plurality of particles are then separated from the biological sample, thereby generating a depleted sample from the biological sample. A biomolecule is purified from the depleted sample, using high-performance liquid chromatography (HPLC) 1220 to produce a purified sample 1230. It is contemplated a higher purity of the biomolecule can be achieved by using the method described above in the purified sample 1230, compared to purifying the biomolecule directly from the biological sample using the HPLC 1220.

It is contemplated that contacting the biological sample with a plurality of particle panels 1210 can deplete high abundant proteins (e.g., albumin, IgG, or a ribosomal protein). In some cases, each particle panel can have one or more nanoparticles. In some cases, the biological sample can be contacted with multiple particle panels 1210 the produce a highly depleted sample. In some case, different particle panels can be used for each round of depletion.

The chromatographic process used herein may utilize one or more chromatographic materials. The chromatographic materials and mobile phase conditions may be of different physical or chemical properties, making the chromatographic process orthogonal. The chromatographic material may comprise hydrophobic interaction, affinity, ion-exchange, mixed mode, reversed phase, size exclusion, and adsorption materials. In some instances, a chromatographic material and mobile phase conditions may be selected to retain particles of a plurality of particles. In other cases, the chromatographic material and mobile phase conditions may be selected to pass through particles of a plurality of particles. In still other cases, the chromatographic material and mobile phase conditions may be selected to retain biomolecules in a sample. In yet other embodiments, the chromatographic materials and mobile phase conditions are selected to pass through biomolecules in a sample. In some cases, the chromatographic process may be a multistep chromatographic process employing two or more chromatographic steps. In some cases, a sequence of the multistep chromatographic process may be designed to separate particles from a plurality of particles.

The methods disclosed herein may also comprise contacting a solution containing particles with a chromatographic material. In one case, the chromatographic material may comprise chromatographic resin in solution, chromatographic resin in a column or chromatographic functionality incorporated into a membrane or onto a surface. The chromatographic material may comprise, but not limited to, ion-exchange, affinity, hydrophobic interaction, mixed mode, reversed phase, size exclusion, and adsorption materials. The adsorption material may be a resin or membrane. In another instances, the chromatographic material may comprise an ion exchanger, a hydrophobic interaction material, a reverse phase material, or a size exclusion material. In one instance, the ionic strength of a particle containing solution may be adjusted. In one instance, the pH of a particle containing solution may be adjusted.

In some cases, sample depletion may be performed by contacting the biofluid with a first particle type, allowing biological molecules to bind to the first particle type, and separating the first particle type along with the bound biological molecules from the biofluid. For example, the particles may be separated from the biofluid using centrifugation or magnetic separation. The biofluid may be further depleted by contacting the fluid with a second particle type, allowing biological molecules to bind to the second particle type, and separating the second particle type along with the bound biological molecules from the biofluid. Depletion may be performed on a biofluid by contacting the biofluid with different particle types and separating each particle type before contacting the biofluid with the next particle type, in a round-by-round manner. In some instances, depletion may be performed with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 particle types. In some instances, sample depletion may be performed using chromatography or electrophoresis.

Depleting a sample may comprise separating and undesired component from a desired component (e.g., separating a high abundance protein from a low abundance protein). In some embodiments, components may be separated based on a biochemical property (e.g., binding affinity, charge, or size). In some instances, depletion may be performed using chromatography or gel electrophoresis. In some cases, a sample may be separated into two or more fractions based on a biochemical property. A first fraction may comprise a desired component, and a second fraction may comprise an undesired component. Fractions comprising desired components may be further processed (e.g., by serial interrogation with various particle types and corona analysis, further fractionation, or depletion). Fractions comprising undesired components may be discarded. Non-limiting examples of depletion methods may include ion-exchange chromatography, affinity chromatography, size exclusion chromatography, gel electrophoresis, or reverse phase chromatography. While these methods are listed by way of example, one of skill in the art could envision numerous other methods that may be used for sample depletion.

Using the compositions and methods disclosed herein, depletion can result in removal of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.9% of high abundance proteins from a biological sample. In some cases, the methods of depletion disclosed herein may remove close to 100% of a high abundance protein.

In some cases, chromatographically purifying a biomolecule from the depleted sample may produce a purified sample. In some instances, the purified sample may have a higher purity of the biomolecule than a sample chromatographically purified directly from the biological sample. In some cases, the purified sample may have at least 10% higher, at least 20% higher, at least 30% higher, at least 40% higher, at least 50% higher, at least 60% higher, at least 70% higher, at least 80% higher, at least 90% higher, or at least 100% higher purity of the biomolecule than a sample chromatographically purified directly from the biological sample.

The separation method described herein may optionally be combined with other separation steps comprising, but not limited to, Protein A chromatography, affinity chromatography, hydrophobic interaction chromatography, immobilized metal affinity chromatography, size exclusion chromatography, diafiltration, ultrafiltration, viral removal filtration, and/or ion exchange chromatography.

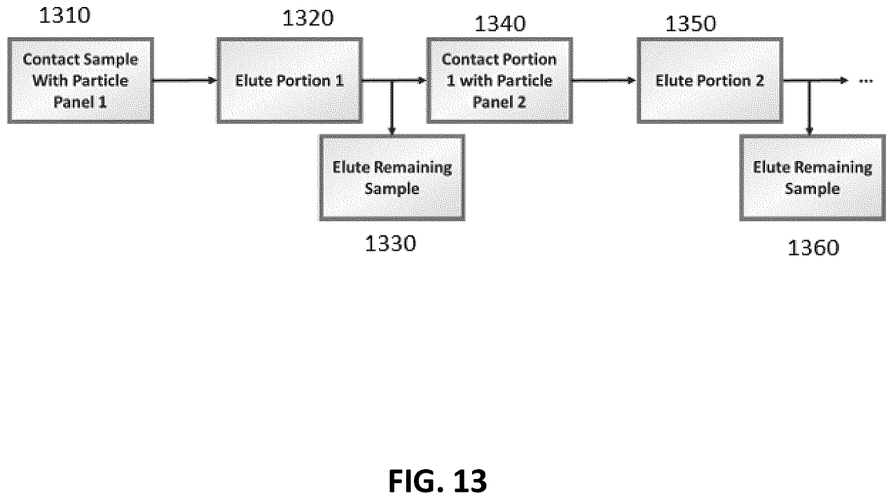

Serial Enrichment. FIG. 13 describes a method of serial enrichment of a biological sample. In Step 1310, a sample is contacted with a Particle Panel 1 (comprising a first particle) under a first condition, thereby adsorbing a first plurality of biomolecules from the biological sample to a surface of the first particle. In Step 1320, Portion 1 (i.e., a first enriched sample) comprising a subset of the first plurality of biomolecules is eluted from Particle Panel 1 and collected, thereby generating the first enriched sample comprising at least a subset of the first plurality of biomolecules. In Step 1340, Portion 1 (i.e., a first enriched sample) is further contacted with Particle Panel 2 (comprising a second particle) under a second condition, thereby adsorbing at least a second plurality of biomolecules from the first enriched sample. In Step 1350, Portion 2 (i.e., a second enriched sample) comprising a subset of the second plurality of biomolecules is eluted from Particle Panel 2 and collected, thereby generating the second enriched sample comprising at least a subset of the second plurality of biomolecules. Further steps can be performed in a similar manner for any desired times.

In some instances, serial enrichment may be performed by sequentially contacting a biological sample with the same or different particle types, allowing biological molecules to bind to the particle type, and separating each particle type and the bound biological molecules before adding the next particle type. Each separated particle type and bound biological molecules may constitute a fraction. In some cases, serial enrichment may be performed with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 particle types. In some cases, serial enrichment with a particle type may be repeated using the sample particle type. In some instances, corona analysis may be performed on a fraction of the serially enriched biological sample. In some cases, corona analysis may be performed on a remaining supernatant of the biological sample following serial enrichment.

The serial enrichment methods disclosed herein may increase the detection of low abundance proteins by protein corona analysis. For example, a serial enrichment method may comprise depleting a sample of high abundance proteins that impact the ability to assay for low abundance proteins. The serial enrichment methods disclosed herein may increase the detection of low abundance proteins by protein corona analysis, because the compressed dynamic range of proteins captured in the protein corona allows for locally concentrating a low abundance protein on the particle surface instead of sampling for the low abundance protein directly in the sample where confounding high abundance proteins are present. In another example, a serial enrichment method may comprise enriching low abundance proteins in biomolecule corona formed on particles. The methods disclosed herein for serially enriching a sample with various sample types may be preceded by depletion or fractionation.

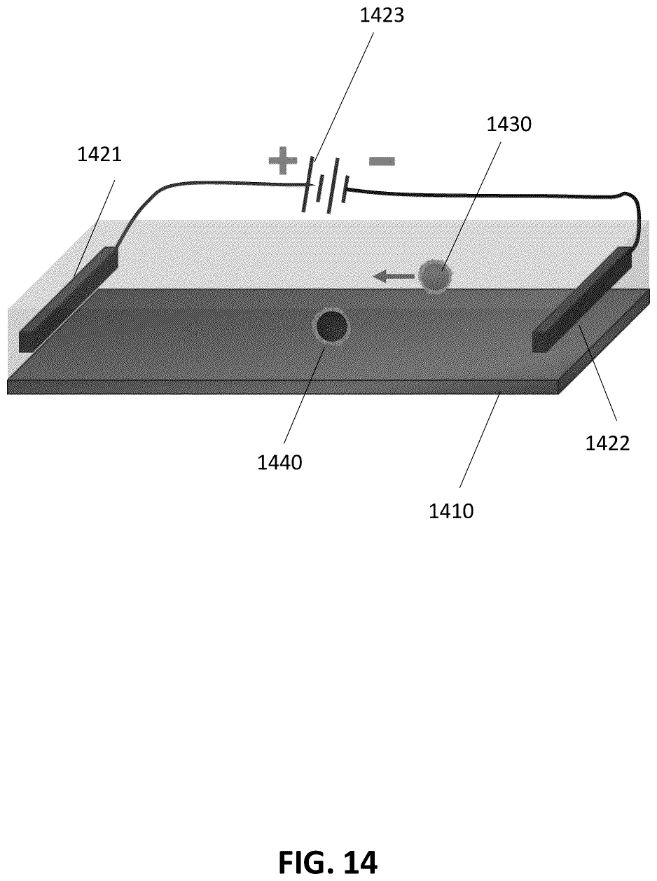

Electrophoretic Particle Separation. FIG. 14 provides a method of fractionating a biological sample using electrophoretic separation of particles. A biological sample is contacted with a plurality of particles, for example, a first particle 1430 and a second particle 1440, allowing biomolecules to adsorb onto the particles and to form biomolecular coronas are formed on the surfaces of the particles. It is contemplated that the first particle 1430 and the second particle 1440 differ in at least one physiochemical property, that different biomolecules adsorb to the two particles, and that the biomolecular coronas formed on the two particles are different. Particles 1430 and 1440 are then separated based at least in part on their zeta potentials. For example, particle 1430 migrates more slowly than the particle 1440 under an electric field, allowing separation of these two particles. The electric field can be formed using a voltage source 1423 and electrodes 1421 and 1422. The separated particles can be collected, and the biomolecules adsorbed to the particles can be desorbed, thereby fractionating the biological sample. As used herein, the term “zeta potential” refers to the surface charge of a nanoparticle in solution, which is the charge that develops at the interface between a solid surface and its liquid medium. Particles with different zeta potentials migrate in different flow directions or speeds in a fluidic system under an electric field. In some cases, the adsorption of biomolecules result in a change in zeta potentials of the particles. In some cases, the adsorption may increase the zeta potentials of certain particles, while in other cases, the adsorption may decrease the zeta potentials of certain particles, depending on the type of biomolecules adsorbed by the particles.

It is further contemplated that electrophoretic separation of particles can be performed by capillary electrophoresis, gel electrophoresis (e.g., 1D or 2D SDS-PAGE or agarose gel), isoelectric focusing (IEF), zone electrophoresis (ZE) and isotachophoresis (ITP), free flow electrophoresis (FFE) or nanofluidics. The electrophoretic force is proportional to the electrophoretic mobility, thus charged particles can be separated based on electrophoretic mobility. In some cases, the separation may require the incorporation of a 2D porous gel. In some cases, separation may require the incorporation of a 3D porous gel. In some cases, microchannels or nanochannels may be applied. In some cases, microslits or nanoslits may be applied. In some cases, the separation may depend on charge-dependent ion mobilities. In some cases, the separation may be dependent on ion valence, zeta potential, ion mobility, or Debye length.

The separation may comprise buffer systems which refer to a mixture of mono, di- or tri-protic/basic compounds. The buffer systems are able to maintain a solution at an essentially constant pH value upon addition of small amounts of acid or base, or upon dilution. A pH distribution may be formed over the entire separation space between the anode and cathode of an apparatus. The buffer systems may be selected from a group comprising, but not limited to, MES/glycylglycine, HEPES/EACA (¿-aminocaproic acid), MES/piperidine-3-carbonic acid, MOPSO/piperidine-4-carbonic acid, and commercially available ampholytes (for example sold under the name Servalyt® by Serva Electrophoresis GmbH, Germany), complementary multi-pair buffer systems (e.g., BD FFE Separation Buffers 1 and 2 sold by BD GmbH, Germany), volatile buffer systems, and binary buffer systems known as A/B media. The anodic and cathodic inter-electrode stabilizing medium may comprise a monoprotic acid and/or a monobasic base. Those of skill in the art may appreciate that the ions formed in the inter electrode stabilizing media may have sufficiently low electrophoretic mobilities. In some case, binary buffer systems may be used for the separation. Suitable buffer bases for the binary buffer systems may comprise taurine, glycine, 2-amino-butyric acid, glycylglycine, β-alanine, GABA, EACA, creatinine, pyridine-ethanol, pyridine-propanol, histidine, BISTRIS, morpholinoethanol, triethanolamine, TRIS, ammediol, benzylamine, diethylaminoethanol, trialkylamines, and the like. Suitable buffer acids may comprise HIBA, acetic acid, picolinic acid, PES, MES, ACES, MOPS, HEPES, EPPS, TAPS, AMPSO, CAPSO, α-alanine, GABA, EACA, 4-hydroxypyridine, 2-hydroxypyridine, and the like, provided the pKa relationships between the buffer acid and buffer base is met.

In some cases, the method may need a viscosity enhancer. The viscosity enhancer is selected from a group comprising polyalcohols (e.g., glycerol or the various PEGs), hydrophilic polymers (e.g., HPMC), carbohydrates (e.g., sucrose, hyaluronic acid). Viscosity enhancers may be required to adapt the viscosity of the medium to the viscosity of the sample introduced into the separation space, or to the viscosity of other separation and/or stabilizing media within the separation chamber in order to avoid turbulences created by the density or viscosity differences between sample and medium or between different adjacent media.

In some cases, the separation may be used to remove all or a portion of a certain protein, in order to enable better access to low abundant proteins or analytes in a sample. In some cases, the separation may be used to selectively separate/isolate high abundant proteins, thereby enabling the analysis of the non-depleted proteins in their native state that have been masked by high abundant proteins. When high abundance proteins are removed or depleted, the relative concentration of other proteins increases. This may be evident as displayed on gel-like images, and as electropherograms for each sample in a tabular format.

### Particle Properties and Types

Particle types consistent with the methods disclosed herein can be made from various materials. For example, particle materials consistent with the present disclosure include metals, polymers, magnetic materials, and lipids. Magnetic particles may be iron oxide particles. Examples of metal materials include any one of or any combination of gold, silver, copper, nickel, cobalt, palladium, platinum, iridium, osmium, rhodium, ruthenium, rhenium, vanadium, chromium, manganese, niobium, molybdenum, tungsten, tantalum, iron and cadmium, or any other material described in U.S. Pat. No. 7,749,299, which is incorporated by reference herein in its entirety. A particle consistent with the compositions and methods disclosed herein may be a magnetic particle, such as a superparamagnetic iron oxide nanoparticle (SPION). A magnetic particle may be a ferromagnetic particle, a ferrimagnetic particle, a paramagnetic particle, a superparamagnetic particle, or any combination thereof (e.g., a particle may comprise a ferromagnetic material and a ferrimagnetic material). A particle may comprise a distinct core (e.g., the innermost portion of the particle), shell (e.g., the outermost layer of the particle), and shell or shells (e.g., portions of the particle disposed between the core and the shell). A particle may comprise a uniform composition.

A particle may comprise a polymer. The polymer may constitute a core material (e.g., the core of a particle may comprise a particle), a layer (e.g., a particle may comprise a layer of a polymer disposed between its core and its shell), a shell material (e.g., the surface of the particle may be coated with a polymer), or any combination thereof. Examples of polymers include any one of or any combination of polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, or polyamines, a polyalkylene glycol (e.g., polyethylene glycol (PEG)), a polyester (e.g., poly(lactide-co-glycolide) (PLGA), polylactic acid, or polycaprolactone), or a copolymer of two or more polymers, such as a copolymer of a polyalkylene glycol (e.g., PEG) and a polyester (e.g., PLGA). The polymer may comprise a cross link. A plurality of polymers in a particle may be phase separated, or may comprise a degree of phase separation. The polymer may comprise a lipid-terminated polyalkylene glycol and a polyester, or any other material disclosed in U.S. Pat. No. 9,549,901 and incorporated by reference herein in its entirety.

In some cases, the liposome comprises a cationic lipid. As used herein, the term “cationic lipid” refers to a lipid that is cationic or becomes cationic (protonated) as the pH is lowered below the pK of the ionizable group of the lipid, but is progressively more neutral at higher pH values. At pH values below the pK, the lipid is then able to associate with negatively charged nucleic acids. In certain embodiments, the cationic lipid comprises a zwitterionic lipid that assumes a positive charge on pH decrease. In certain embodiments, the liposomes comprise cationic lipid. In some embodiments, cationic lipid comprises any of a number of lipid species which carry a net positive charge at a selective pH, such as physiological pH. Such lipids include, but are not limited to, N,N-dioleyl-N,N-dimethylammonium chloride (DODAC); N-(2,3-dioleyloxy) propyl)-N,N,N-trimethylammonium chloride (DOTMA); N,N-distearyl-N,N-dimethylammonium bromide (DDAB); N-(2,3-dioleoyloxy) propyl)-N,N,N-trimethylammonium chloride (DOTAP); 3-(N—(N′,N′-dimethylaminoethane)-carbamoyl) cholesterol (DC-Chol), N-(1-(2,3-dioleoyloxy) propyl)-N-2-(sperminecarboxamido)ethyl)-N,N-dimethylammonium trifluoracetate (DOSPA), dioctadecylamidoglycyl carboxyspermine (DOGS), 1,2-dioleoyl-3-dimethylammonium propane (DODAP), N,N-dimethyl-2,3-dioleoyloxy) propylamine (DODMA), N-(1,2-dimyristyloxyprop-3-yl)-N,N-dimethyl-N-hydroxyethyl ammonium bromide (DMRIE), 1,2-dioleoyl-sn-3-phosphoethanolamine (DOPE), N-(1-(2,3-dioleyloxy) propyl)-N-(2-(sperminecarboxamido)ethyl)-N,N-dimethy-lammonium trifluoroacetate (DOSPA), dioctadecylamidoglycyl carboxyspermine (DOGS), and 1,2-ditetradecanoyl-sn-glycero-3-phosphocholine (DMPC). The following lipids are cationic and have a positive charge at below physiological pH: DODAP, DODMA, DMDMA, 1,2-dilinoleyloxy-N,N-dimethylaminopropane (DLinDMA), 1,2-dilinolenyloxy-N,N-dimethylaminopropane (DLenDMA, or any other lipid disclosed in U.S. Pat. No. 10,866,242, which is incorporated by reference herein in its entirety.

In some cases, examples of lipids that can be used to form the particles of the present disclosure include cationic, anionic, and neutrally charged lipids. For example, particles can be made of any one of or any combination of dioleoylphosphatidylglycerol (DOPG), diacylphosphatidylcholine, diacylphosphatidylethanolamine, ceramide, sphingomyelin, cephalin, cholesterol, cerebrosides and diacylglycerols, dioleoylphosphatidylcholine (DOPC), dimyristoylphosphatidylcholine (DMPC), and dioleoylphosphatidylserine (DOPS), phosphatidylglycerol, cardiolipin, diacylphosphatidylserine, diacylphosphatidic acid, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), lecithin, lysolecithin, phosphatidylethanolamine, lysophosphatidylethanolamine, dioleoylphosphatidylethanolamine (DOPE), dipalmitoyl phosphatidyl ethanolamine (DPPE), dimyristoylphosphoethanolamine (DMPE), distearoyl-phosphatidyl-ethanolamine (DSPE), palmitoyloleoyl-phosphatidylethanolamine (POPE) palmitoyloleoylphosphatidylcholine (POPC), egg phosphatidylcholine (EPC), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), palmitoyloleyolphosphatidylglycerol (POPG), 16-O-monomethyl PE, 16-O-dimethyl PE, 18-1-trans PE, palmitoyloleoyl-phosphatidylethanolamine (POPE), 1-stearoyl-2-oleoyl-phosphatidyethanolamine (SOPE), phosphatidylserine, phosphatidylinositol, sphingomyelin, cephalin, cardiolipin, phosphatidic acid, cerebrosides, dicetylphosphate, and cholesterol, or any other material listed in U.S. Pat. No. 9,445,994, which is incorporated by reference herein in its entirety.

Examples of particles of the present disclosure are provided in TABLE 1.

TABLE 1
Example particles of the present disclosure

| Batch No. | Type | Particle ID | Description |
|---|---|---|---|
| S-001-001 | HX-13 | SP-001 | Carboxylate (Citrate) superparamagnetic iron oxide NPs |
| | | | (SPION) |
| S-002-001 | HX-19 | SP-002 | Phenol-formaldehyde coated SPION |
| S-003-001 | HX-20 | SP-003 | Silica-coated superparamagnetic iron oxide NPs |
| | | | (SPION) |
| S-004-001 | HX-31 | SP-004 | Polystyrene coated SPION |
| S-005-001 | HX-38 | SP-005 | Carboxylated Poly(styrene-co-methacrylic acid), P(St- |
| | | | co-MAA) coated SPION |
| S-006-001 | HX-42 | SP-006 | N-(3-Trimethoxysilylpropyl)diethylenetriamine coated |
| | | | SPION |
| S-007-001 | HX-56 | SP-007 | poly(N-(3-(dimethylamino)propyl) methacrylamide) |
| | | | (PDMAPMA)-coated SPION |
| S-008-001 | HX-57 | SP-008 | 1,2,4,5-Benzenetetracarboxylic acid coated SPION |
| S-009-001 | HX-58 | SP-009 | PVBTMAC coated |
| | | | poly(vinylbenzyltrimethylammonium chloride) |
| | | | (PVBTMAC) coated SPION |
| S-010-001 | HX-59 | SP-010 | Carboxylate, PAA coated SPION |
| S-011-001 | HX-86 | SP-011 | poly(oligo(ethylene glycol) methyl ether methacrylate) |
| | | | (POEGMA)-coated SPION |
| S-163-001 | | S-163 | Cis-ubiquitin-functionalized styrene particle |
| S-164-001 | | S-164 | Ubiquitin-functionalized styrene particle |
| P-033-001 | P33 | SP-333 | Carboxylate functionalized 1 μm magnetic |
| | | | microparticle, surfactant free SPION |
| P-039-003 | P39 | SP-339 | Polystyrene carboxyl functionalized SPION |
| P-041-001 | P41 | SP-341 | Carboxylic acid SPION |
| P-047-001 | P47 | SP-365 | Silica SPION |
| P-048-001 | P48 | SP-348 | Carboxylic acid, 150 nm SPION |
| P-053-001 | P53 | SP-353 | Amino surface microparticle, 0.4-0.6 μm SPION |
| P-056-001 | P56 | SP-356 | Silica amino functionalized microparticle, 0.1-0.39 μm |
| | | | SPION |
| P-063-001 | P63 | SP-363 | Jeffamine surface, 0.1-0.39 μm SPION |
| P-064-001 | P64 | SP-364 | Polystyrene microparticle, 2.0-2.9 μm SPION |
| P-065-001 | P65 | SP-365 | Silica SPION |
| P-069-001 | P69 | SP-369 | Carboxylated Original coating, 50 nm SPION |
| P-073-001 | P73 | SP-373 | Dextran based coating, 0.13 μm SPION |
| P-074-001 | P74 | SP-374 | Silica Silanol coated with lower acidity SPION |
| — | S-118 | SP-118 | 1,6-hexanediamine functionalized SPION |
| — | S-125 | SP-125 | Amine functionalized silica-coated SPION |
| — | S-128 | SP-128 | Mixed amide, carboxylate functionalized, silica-coated |
| | | | SPION |
| — | S-199 | SP-199 | Epichlorohydrin crosslinked Dextran-coated SPION |
| — | S-229 | SP-229 | N¹-(3-(trimethoxysilyl)propyl)hexane-1,6-diamine |
| | | | functionalized, silica-coated SPION |

A particle of the present disclosure may be synthesized, or a particle of the present disclosure may be purchased from a commercial vendor. For example, particles consistent with the present disclosure may be purchased from commercial vendors including Sigma-Aldrich, Life Technologies, Fisher Biosciences, nanoComposix, Nanopartz, Spherotech, and other commercial vendors. In some cases, a particle of the present disclosure may be purchased from a commercial vendor and further modified, coated, or functionalized.

An example of a particle type of the present disclosure may be a carboxylate (Citrate) superparamagnetic iron oxide nanoparticle (SPION), a phenol-formaldehyde coated SPION, a silica-coated SPION, a polystyrene coated SPION, a carboxylated poly(styrene-co-methacrylic acid) coated SPION, a N-(3-Trimethoxysilylpropyl) diethylenetriamine coated SPION, a poly(N-(3-(dimethylamino) propyl) methacrylamide) (PDMAPMA)-coated SPION, a 1,2,4,5-Benzenetetracarboxylic acid coated SPION, a poly(Vinylbenzyltrimethylammonium chloride) (PVBTMAC) coated SPION, a carboxylate, PAA coated SPION, a poly(oligo (ethylene glycol) methyl ether methacrylate) (POEGMA)-coated SPION, a carboxylate microparticle, a polystyrene carboxyl functionalized particle, a carboxylic acid coated particle, a silica particle, a carboxylic acid particle of about 150 nm in diameter, an amino surface microparticle of about 0.4-0.6 μm in diameter, a silica amino functionalized microparticle of about 0.1-0.39 μm in diameter, a Jeffamine surface particle of about 0.1-0.39 μm in diameter, a polystyrene microparticle of about 2.0-2.9 μm in diameter, a silica particle, a carboxylated particle with an original coating of about 50 nm in diameter, a particle coated with a dextran based coating of about 0.13 μm in diameter, or a silica silanol coated particle with low acidity.

A particle may be provided at a range of concentrations. A particle may comprise a concentration between 100 fM and 100 nM. A particle may comprise a concentration between 100 fM and 10 pM. A particle may comprise a concentration between 1 pM and 100 pM. A particle may comprise a concentration between 10 pM and 1 nM. A particle may comprise a concentration between 100 pM and 10 nM. A particle may comprise a concentration between 1 nM and 100 nM. A particle may be contacted to a biological sample at a ratio of volume ratios. A solution comprising a particle may be combined with a biological sample, at a volume ratio of greater than about 100:1, about 100:1, about 80:1, about 60:1, about 50:1, about 40:1, about 30:1, about 25:1, about 20:1, about 15:1, about 12:1, about 10:1, about 8:1, about 6:1, about 5:1, about 4:1, about 3:1, about 5:2, about 2:1, about 3:2, about 1:1, about 2:3, about 1:2, about 2:5, about 1:3, about 1:4, about 1:5, about 1:6, about 1:8, about 1:10, about 1:12, about 1:15, about 1:20, about 1:25, about 1:30, about 1:40, about 1:50, about 1:60, about 1:80, about 1:100, or less than about 1:100.

Particles that are consistent with the present disclosure can comprise a wide range of sizes. In some cases, a particle of the present disclosure may be a nanoparticle. In some cases, a nanoparticle of the present disclosure may be from about 10 nm to about 1000 nm in diameter. For example, the nanoparticles disclosed herein can be at least 10 nm, at least 100 nm, at least 200 nm, at least 300 nm, at least 400 nm, at least 500 nm, at least 600 nm, at least 700 nm, at least 800 nm, at least 900 nm, from 10 nm to 50 nm, from 50 nm to 100 nm, from 100 nm to 150 nm, from 150 nm to 200 nm, from 200 nm to 250 nm, from 250 nm to 300 nm, from 300 nm to 350 nm, from 350 nm to 400 nm, from 400 nm to 450 nm, from 450 nm to 500 nm, from 500 nm to 550 nm, from 550 nm to 600 nm, from 600 nm to 650 nm, from 650 nm to 700 nm, from 700 nm to 750 nm, from 750 nm to 800 nm, from 800 nm to 850 nm, from 850 nm to 900 nm, from 100 nm to 300 nm, from 150 nm to 350 nm, from 200 nm to 400 nm, from 250 nm to 450 nm, from 300 nm to 500 nm, from 350 nm to 550 nm, from 400 nm to 600 nm, from 450 nm to 650 nm, from 500 nm to 700 nm, from 550 nm to 750 nm, from 600 nm to 800 nm, from 650 nm to 850 nm, from 700 nm to 900 nm, or from 10 nm to 900 nm in diameter. In some cases, a nanoparticle may be less than 1000 nm in diameter.

A particle of the present disclosure may be a microparticle. A microparticle may be a particle that is from about 1 μm to about 1000 μm in diameter. For example, the microparticles disclosed here can be at least 1 μm, at least 10 μm, at least 100 μm, at least 200 μm, at least 300 μm, at least 400 μm, at least 500 μm, at least 600 μm, at least 700 μm, at least 800 μm, at least 900 μm, from 10 μm to 50 μm, from 50 μm to 100 μm, from 100 μm to 150 μm, from 150 μm to 200 μm, from 200 μm to 250 μm, from 250 μm to 300 μm, from 300 μm to 350 μm, from 350 μm to 400 μm, from 400 μm to 450 μm, from 450 μm to 500 μm, from 500 μm to 550 μm, from 550 μm to 600 μm, from 600 μm to 650 μm, from 650 μm to 700 μm, from 700 μm to 750 μm, from 750 μm to 800 μm, from 800 μm to 850 μm, from 850 μm to 900 μm, from 100 μm to 300 μm, from 150 μm to 350 μm, from 200 μm to 400 μm, from 250 μm to 450 μm, from 300 μm to 500 μm, from 350 μm to 550 μm, from 400 μm to 600 μm, from 450 μm to 650 μm, from 500 μm to 700 μm, from 550 μm to 750 μm, from 600 μm to 800 μm, from 650 μm to 850 μm, from 700 μm to 900 μm, or from 10 μm to 900 μm in diameter. In some cases, a microparticle may be less than 1000 μm in diameter.

The ratio between surface area and mass can be a determinant of a particle's properties in the methods of the instant disclosure. For example, the number and types of biomolecules that a particle adsorbs from a solution may vary with the particle's surface area to mass ratio. The particles disclosed herein can have surface area to mass ratios of 3 to 30 cm²/mg, 5 to 50 cm²/mg, 10 to 60 cm²/mg, 15 to 70 cm²/mg, 20 to 80 cm²/mg, 30 to 100 cm²/mg, 35 to 120 cm²/mg, 40 to 130 cm²/mg, 45 to 150 cm²/mg, 50 to 160 cm²/mg, 60 to 180 cm²/mg, 70 to 200 cm²/mg, 80 to 220 cm²/mg, 90 to 240 cm²/mg, 100 to 270 cm²/mg, 120 to 300 cm²/mg, 200 to 500 cm²/mg, 10 to 300 cm²/mg, 1 to 3000 cm²/mg, 20 to 150 cm²/mg, 25 to 120 cm²/mg, or from 40 to 85 cm²/mg. Small particles (e.g., with diameters of 50 nm or less) can have higher surface area to mass ratios than large particles (e.g., with diameters of 200 nm or more) . . . . In some cases (e.g., for small particles), the particles can have surface area to mass ratios of 200 to 1000 cm²/mg, 500 to 2000 cm²/mg, 1000 to 4000 cm²/mg, 2000 to 8000 cm²/mg, or 4000 to 10000 cm²/mg. In some cases (e.g., for large particles), the particles can have surface area to mass ratios of 1 to 3 cm²/mg, 0.5 to 2 cm²/mg, 0.25 to 1.5 cm²/mg, or 0.1 to 1 cm²/mg.

In some cases, a plurality of particles (e.g., of a particle panel) of the compositions and methods described herein may comprise a range of surface area to mass ratios. In some cases, the range of surface area to mass ratios for a plurality of particles is less than 100 cm²/mg, 80 cm²/mg, 60 cm²/mg, 40 cm²/mg, 20 cm²/mg, 10 cm²/mg, 5 cm²/mg, or 2 cm²/mg. In some cases, the surface area to mass ratios for a plurality of particles varies by no more than 40%, 30%, 20%, 10%, 5%, 3%, 2%, or 1% between the particles in the plurality.

In some cases, a plurality of particles (e.g., particles having the same or different physicochemical properties) may have a wider range of surface area to mass ratios. In some cases, the range of surface area to mass ratios for a plurality of particles is greater than 100 cm²/mg, 150 cm²/mg, 200 cm²/mg, 250 cm²/mg, 300 cm²/mg, 400 cm²/mg, 500 cm²/mg, 800 cm²/mg, 1000 cm²/mg, 1200 cm²/mg, 1500 cm²/mg, 2000 cm²/mg, 3000 cm²/mg, 5000 cm²/mg, 7500 cm²/mg, 10000 cm²/mg, or more. In some cases, the surface area to mass ratios for a plurality of particles (e.g., within a panel) can vary by more than 100%, 200%, 300%, 400%, 500%, 1000%, 10000% or more. In some cases, the plurality of particles with a wide range of surface area to mass ratios comprises at least 2, 3, 4, 5, 6, 7, 8, 9, 10, 12, 15, 20, or more different types of particles.

A particle may comprise a wide array of physical properties. A physical property of a particle may include composition, size, surface charge, hydrophobicity, hydrophilicity, surface shape, and any combination thereof.

A surface functionalization may comprise a polymerizable functional group, a positively or negatively charged functional group, a zwitterionic functional group, an acidic or basic functional group, a polar functional group, or any combination thereof. A surface functionalization may comprise carboxyl groups, hydroxyl groups, thiol groups, cyano groups, nitro groups, ammonium groups, alkyl groups, imidazolium groups, sulfonium groups, pyridinium groups, pyrrolidinium groups, phosphonium groups, aminopropyl groups, amine groups, boronic acid groups, N-succinimidyl ester groups, PEG groups, streptavidin, methyl ether groups, triethoxylpropylaminosilane groups, PCP groups, citrate groups, lipoic acid groups, BPEI groups, or any combination thereof. A particle from among the plurality of particles may be selected from the group consisting of: micelles, liposomes, iron oxide particles, silver particles, gold particles, palladium particles, quantum dots, platinum particles, titanium particles, silica particles, metal or inorganic oxide particles, synthetic polymer particles, copolymer particles, terpolymer particles, polymeric particles with metal cores, polymeric particles with metal oxide cores, polystyrene sulfonate particles, polyethylene oxide particles, polyoxyethylene glycol particles, polyethylene imine particles, polylactic acid particles, polycaprolactone particles, polyglycolic acid particles, poly(lactide-co-glycolide polymer particles, cellulose ether polymer particles, polyvinylpyrrolidone particles, polyvinyl acetate particles, polyvinylpyrrolidone-vinyl acetate copolymer particles, polyvinyl alcohol particles, acrylate particles, polyacrylic acid particles, crotonic acid copolymer particles, polyethlene phosphonate particles, polyalkylene particles, carboxy vinyl polymer particles, sodium alginate particles, carrageenan particles, xanthan gum particles, gum acacia particles, Arabic gum particles, guar gum particles, pullulan particles, agar particles, chitin particles, chitosan particles, pectin particles, karaya tum particles, locust bean gum particles, maltodextrin particles, amylose particles, corn starch particles, potato starch particles, rice starch particles, tapioca starch particles, pea starch particles, sweet potato starch particles, barley starch particles, wheat starch particles, hydroxypropylated high amylose starch particles, dextrin particles, levan particles, elsinan particles, gluten particles, collagen particles, whey protein isolate particles, casein particles, milk protein particles, soy protein particles, keratin particles, polyethylene particles, polycarbonate particles, polyanhydride particles, polyhydroxyacid particles, polypropylfumerate particles, polycaprolactone particles, polyamine particles, polyacetal particles, polyether particles, polyester particles, poly(orthoester) particles, polycyanoacrylate particles, polyurethane particles, polyphosphazene particles, polyacrylate particles, polymethacrylate particles, polycyanoacrylate particles, polyurea particles, polyamine particles, polystyrene particles, poly(lysine) particles, chitosan particles, dextran particles, poly(acrylamide) particles, derivatized poly(acrylamide) particles, gelatin particles, starch particles, chitosan particles, dextran particles, gelatin particles, starch particles, poly-β-amino-ester particles, poly(amido amine) particles, poly lactic-co-glycolic acid particles, polyanhydride particles, bioreducible polymer particles, and 2-(3-aminopropylamino) ethanol particles, and any combination thereof.

Particles of the present disclosure may differ by one or more physicochemical property. The one or more physicochemical property is selected from the group consisting of: composition, size, surface charge, hydrophobicity, hydrophilicity, roughness, density surface functionalization, surface topography, surface curvature, porosity, core material, shell material, shape, and any combination thereof. The surface functionalization may comprise a macromolecular functionalization, a small molecule functionalization, or any combination thereof. A small molecule functionalization may comprise an aminopropyl functionalization, amine functionalization, boronic acid functionalization, carboxylic acid functionalization, alkyl group functionalization, N-succinimidyl ester functionalization, monosaccharide functionalization, phosphate sugar functionalization, sulfurylated sugar functionalization, ethylene glycol functionalization, streptavidin functionalization, methyl ether functionalization, trimethoxysilylpropyl functionalization, silica functionalization, triethoxylpropylaminosilane functionalization, thiol functionalization, PCP functionalization, citrate functionalization, lipoic acid functionalization, ethyleneimine functionalization. A particle panel may comprise a plurality of particles with a plurality of small molecule functionalizations selected from the group consisting of silica functionalization, trimethoxysilylpropyl functionalization, dimethylamino propyl functionalization, phosphate sugar functionalization, amine functionalization, and carboxyl functionalization.

A small molecule functionalization may comprise a polar functional group. Non-limiting examples of polar functional groups comprise carboxyl group, a hydroxyl group, a thiol group, a cyano group, a nitro group, an ammonium group, an imidazolium group, a sulfonium group, a pyridinium group, a pyrrolidinium group, a phosphonium group or any combination thereof. In some embodiments, the functional group is an acidic functional group (e.g., sulfonic acid group, carboxyl group, and the like), a basic functional group (e.g., amino group, cyclic secondary amino group (such as pyrrolidyl group and piperidyl group), pyridyl group, imidazole group, guanidine group, etc.), a carbamoyl group, a hydroxyl group, an aldehyde group and the like.

A small molecule functionalization may comprise an ionic or ionizable functional group. Non-limiting examples of ionic or ionizable functional groups comprise an ammonium group, an imidazolium group, a sulfonium group, a pyridinium group, a pyrrolidinium group, a phosphonium group.

A small molecule functionalization may comprise a polymerizable functional group. Non-limiting examples of the polymerizable functional group include a vinyl group and a (meth)acrylic group. In some embodiments, the functional group is pyrrolidyl acrylate, acrylic acid, methacrylic acid, acrylamide, 2-(dimethylamino)ethyl methacrylate, hydroxyethyl methacrylate and the like.

A surface functionalization may comprise a charge. For example, a particle can be functionalized to carry a net neutral surface charge, a net positive surface charge, a net negative surface charge, or a zwitterionic surface. Surface charge can be a determinant of the types of biomolecules collected on a particle. Accordingly, optimizing a particle panel may comprise selecting particles with different surface charges, which may not only increase the number of different proteins collected on a particle panel, but also increase the likelihood of identifying a biological state of a sample. A particle panel may comprise a positively charged particle and a negatively charged particle. A particle panel may comprise a positively charged particle and a neutral particle. A particle panel may comprise a positively charged particle and a zwitterionic particle. A particle panel may comprise a neutral particle and a negatively charged particle. A particle panel may comprise a neutral particle and a zwitterionic particle. A particle panel may comprise a negative particle and a zwitterionic particle. A particle panel may comprise a positively charged particle, a negatively charged particle, and a neutral particle. A particle panel may comprise a positively charged particle, a negatively charged particle, and a zwitterionic particle. A particle panel may comprise a positively charged particle, a neutral particle, and a zwitterionic particle. A particle panel may comprise a negatively charged particle, a neutral particle, and a zwitterionic particle.

The present disclosure includes compositions (e.g., particles and particle panels) and methods that comprise two or more particles differing in at least one physicochemical property. A composition or method of the present disclosure may comprise 3 to 6 particles differing in at least one physicochemical property. A composition or method of the present disclosure may comprise 4 to 8 particles differing in at least one physicochemical property. A composition or method of the present disclosure may comprise 4 to 10 particles differing in at least one physicochemical property. A composition or method of the present disclosure may comprise 5 to 12 particles differing in at least one physicochemical property. A composition or method of the present disclosure may comprise 6 to 14 particles differing in at least one physicochemical property. A composition or method of the present disclosure may comprise 8 to 15 particles differing in at least one physicochemical property. A composition or method of the present disclosure may comprise 10 to 20 particles differing in at least one physicochemical property. A composition or method of the present disclosure may comprise at least 2 distinct particle types, at least 3 distinct particle types, at least 4 distinct particle types, at least 5 distinct particle types, at least 6 distinct particle types, at least 7 distinct particle types, at least 8 distinct particle types, at least 9 distinct particle types, at least 10 distinct particle types, at least 11 distinct particle types, at least 12 distinct particle types, at least 13 distinct particle types, at least 14 distinct particle types, at least 15 distinct particle types, at least 20 distinct particle types, at least 25 particle types, or at least 30 distinct particle types.

Surface functionalization can influence the composition of a particle's biomolecule corona. Such surface functionalization can include small molecule functionalization or macromolecular functionalization. A surface functionalization may be coupled to a particle material such as a polymer, metal, metal oxide, inorganic oxide (e.g., silicon dioxide), or another surface functionalization.

A surface functionalization may comprise a small molecule functionalization, a macromolecular functionalization, or a combination of two or more such functionalizations. A macromolecular functionalization may comprise a biomacromolecule, such as a protein or a polynucleotide (e.g., a 100-mer DNA molecule). A macromolecular functionalization may be comprise a protein, polynucleotide, or polysaccharide, or may be comparable in size to any of the aforementioned classes of species. For example, a macromolecular functionalization may comprise a volume of at least 6 nm³, at least 8 nm³, at least 12 nm³, at least 15 nm³, at least 20 nm³, at least 30 nm³, at least 50 nm³, at least 80 nm³, at least 120 nm³, at least 180 nm³, at least 300 nm³, at least 500 nm³, at least 800 nm³, at least 1200 nm³, at least 1500 nm³, or at least 2000 nm³. A macromolecular functionalization may comprise a surface area of at least at least 15 nm², at least 20 nm², at least 25 nm², at least 40 nm², at least 80 nm², at least 150 nm², at least 300 nm², at least 500 nm², at least 800 nm², at least 1200 nm², or at least 1500 nm². A macromolecular functionalization may comprise a bait molecule.

A macromolecular functionalization may comprise a specific form of attachment to a particle. A macromolecule may be tethered to a particle via a linker. The linker may hold the macromolecule close to the particle, thereby restricting its motion and reorientation relative to the particle, or may extend the macromolecule away from the particle. The linker may be rigid (e.g., a polyolefin linker) or flexible (e.g., a nucleic acid linker). A linker may be no more than 0.5 nm in length, no more than 1 nm in length, no more than 1.5 nm in length, no more than 2 nm in length, no more than 3 nm in length, no more than 4 nm in length, no more than 5 nm in length, no more than 8 nm in length, or no more than 10 nm in length. A linker may be at least 1 nm in length, at least 2 nm in length, at least 3 nm in length, at least 4 nm in length, at least 5 nm in length, at least 8 nm in length, at least 12 nm in length, at least 15 nm in length, at least 20 nm in length, at least 25 nm in length, or at least 30 nm in length. As such, a surface functionalization on a particle may project beyond a primary corona associated with the particle. A surface functionalization may also be situated beneath or within a biomolecule corona that forms on the particle surface.

A macromolecule may be tethered at a specific location, such as a protein's C-terminus, or may be tethered at a number of possible sites. For example, a peptide may be covalent attached to a particle via any of its surface exposed lysine residues.

A particle may comprise a single surface such as a specific small molecule, or a plurality of surface functionalizations, such as a plurality of different small molecules.

A particle may comprise a high affinity for a particular biomolecule or class of biomolecules. For example, a surface functionalization may comprise a nonpolar moiety (such as an organosilane) that interacts strongly with nonpolar protein functional groups and alpha helices. Analogously, a macromolecular surface functionalization may comprise a peptide (e.g., an antibody) with a high affinity for a specific molecular target.

A particle may comprise a small molecule functionalization. A small molecule functionalization may comprise a mass of fewer than 600 Daltons, fewer than 500 Daltons, fewer than 400 Daltons, fewer than 300 Daltons, fewer than 200 Daltons, or fewer than 100 Daltons. A small molecule functionalization may comprise an ionizable moiety, such as a chemical group with a pKₐ or pK_{b} of less than 6 or 7. A small molecule functionalization may comprise a small organic molecule such as an alcohol (e.g., octanol), an amine, an alkane, an alkene, an alkyne, a heterocycle (e.g., a piperidinyl group), a heteroaromatic group, a thiol, a carboxylate, a carbonyl, an amide, an ester, a thioester, a carbonate, a thiocarbonate, a carbamate, a thiocarbamate, a urea, a thiourea, a halogen, a sulfate, a phosphate, a monosaccharide, a disaccharide, a lipid, or any combination thereof. For example, a small molecule functionalization may comprise a phosphate sugar, a sugar acid, or a sulfurylated sugar.

A particle of the present disclosure may be contacted with a biological sample (e.g., a biofluid) to form a biomolecule corona. The particle and biomolecule corona may be separated from the biological sample, for example by centrifugation, magnetic separation, filtration, or gravitational separation. The particle types and biomolecule corona may be separated from the biological sample using a number of separation techniques. Non-limiting examples of separation techniques include comprises magnetic separation, charge-based separation, column-based separation, filtration, spin column-based separation, centrifugation, ultracentrifugation, density or gradient-based centrifugation, gravitational separation, or any combination thereof. Each of a plurality of particle types may be separated from a mixture of particles based on their physical, chemical, charge, or magnetic properties. A protein corona analysis may be performed on the separated particle and biomolecule corona. A protein corona analysis may comprise identifying one or more proteins in the biomolecule corona, for example by mass spectrometry. A single particle type (e.g., a particle of a type listed in TABLE 1) may be contacted to a biological sample. A plurality of particle types (e.g., a plurality of the particle types provided in TABLE 1) may be contacted to a biological sample. The plurality of particle types may be combined and contacted to the biological sample in a single sample volume. The plurality of particle types may be sequentially contacted to a biological sample and separated from the biological sample prior to contacting a subsequent particle type to the biological sample. Protein corona analysis of the biomolecule corona may compress the dynamic range of the analysis compared to a total protein analysis method.

The particles of the present disclosure may be used to serially interrogate a sample by incubating a first particle type with the sample to form a biomolecule corona on the first particle type, separating the first particle type, incubating a second particle type with the sample to form a biomolecule corona on the second particle type, separating the second particle type, and repeating the interrogating (by incubation with the sample) and the separating for any number of particle types. In some cases, the biomolecule corona on each particle type used for serial interrogation of a sample may be analyzed by protein corona analysis. The biomolecule content of the supernatant may be analyzed following serial interrogation with one or more particle types.

### Particles and Particle Panels

The present disclosure provides compositions and methods of use thereof for assaying a sample for proteins. The methods, compositions and devices disclosed herein may include one or more particles, comprising one or more particle types. In some instances, the methods, compositions and devices described herein may include particle panels comprising more than one distinct particle type. Particle panels described herein can vary in the number of particle types and the diversity of particle types in a single panel. For example, particles in a panel may vary based on size, polydispersity, shape and morphology, surface charge, surface chemistry and functionalization, and base material. Panels may be incubated with a sample to be analyzed for proteins and protein concentrations. Proteins in the sample adsorb to the surface of the different particle types in the particle panel to form a protein corona. The exact protein and the concentration of protein that adsorbs to a certain particle type in the particle panel may depend on the composition, size, and surface charge of the particle type. Thus, each particle type in a panel may have different protein coronas due to adsorbing a different set of proteins, different concentrations of a particular protein, or a combination thereof. Each particle type in a panel may have mutually exclusive protein coronas or may have overlapping protein coronas. Overlapping protein coronas can overlap in protein identity, in protein concentration, or both.

The present disclosure also provides methods for selecting a particle types for inclusion in a panel depending on the sample type. Particle types included in a panel may be a combination of particles that are optimized for removal of highly abundant proteins. Particle types also consistent for inclusion in a panel are those selected for adsorbing particular proteins of interest. The particles can be nanoparticles. The particles can be microparticles. The particles can be a combination of nanoparticles and microparticles.

A particle panel including any number of distinct particle types disclosed herein or disclosed in U.S. Pat. No. 11,428,688, which is incorporated by reference herein in its entirety, enriches and identifies a single protein or protein group. In some cases, the single protein or protein group may comprise proteins having different post-translational modifications. For example, a first particle type in the particle panel may enrich a protein or protein group having a first post-translational modification, a second particle type in the particle panel may enrich the same protein or same protein group having a second post-translational modification, and a third particle type in the particle panel may enrich the same protein or same protein group lacking a post-translational modification. In some cases, the particle panel including any number of distinct particle types disclosed herein, enriches and identifies a single protein or protein group by binding different domains, sequences, or epitopes of the single protein or protein group. For example, a first particle type in the particle panel may enrich a protein or protein group by binding to a first domain of the protein or protein group, and a second particle type in the particle panel may enrich the same protein or same protein group by binding to a second domain of the protein or protein group.

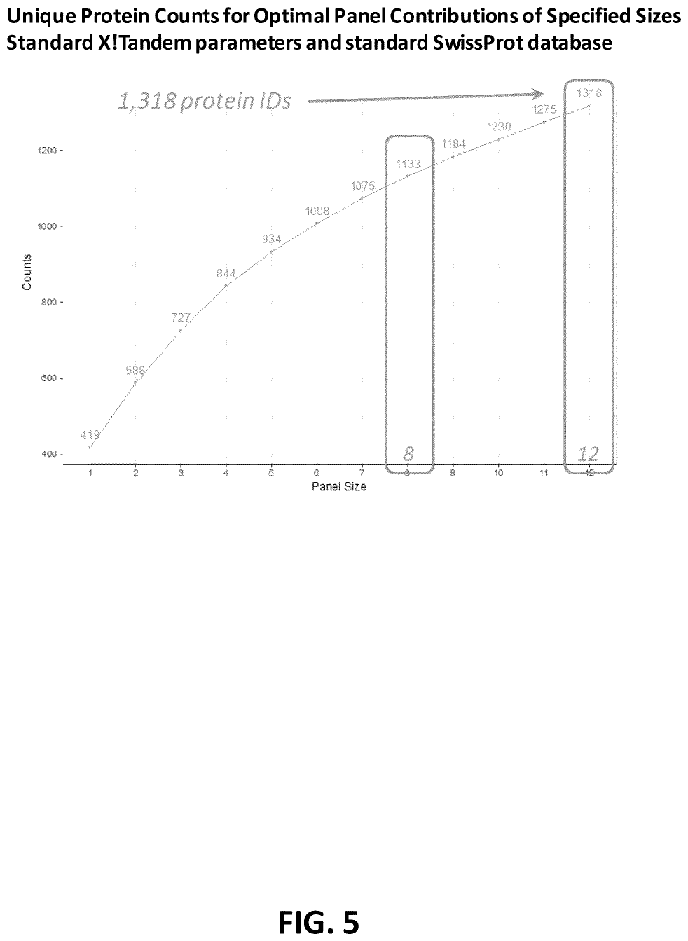

A particle panel can have more than one particle type. Increasing the number of particle types in a panel can be a method for increasing the number of proteins that can be identified in a given sample. An example of how increasing panel size may increase the number of identified proteins is shown in FIG. 5, in which a panel size of one particle type identified 419 different proteins, a panel size of two particle types identified 588 different proteins, a panel size of three particle types identified 727 different proteins, a panel size of four particle types identified 844 proteins, a panel size of five particle types identified 934 different proteins, a panel size of six particle types identified 1008 different proteins, a panel size of seven particle types identified 1075 different proteins, a panel size of eight particle types identified 1133 different proteins, a panel size of nine particle types identified 1184 different proteins, a panel size of 10 particle types identified 1230 different proteins, a panel size of 11 particle types identified 1275 different proteins, and a panel size of 12 particle types identified 1318 different proteins.

A particle panel may comprise a combination of particles with silica and polymer surfaces. For example, a particle panel may comprise a SPION coated with a thin layer of silica, a SPION coated with poly(dimethyl aminopropyl methacrylamide) (PDMAPMA), and a SPION coated with poly(ethylene glycol) (PEG). A particle panel consistent with the present disclosure could also comprise two or more particles selected from the group consisting of silica coated SPION, an N-(3-Trimethoxysilylpropyl) diethylenetriamine coated SPION, a PDMAPMA coated SPION, a carboxyl-functionalized polyacrylic acid coated SPION, an amino surface functionalized SPION, a polystyrene carboxyl functionalized SPION, a silica particle, and a dextran coated SPION. A particle panel consistent with the present disclosure may also comprise two or more particles selected from the group consisting of a surfactant free carboxylate microparticle, a carboxyl functionalized polystyrene particle, a silica coated particle, a silica particle, a dextran coated particle, an oleic acid coated particle, a boronated nanopowder coated particle, a PDMAPMA coated particle, a Poly(glycidyl methacrylate-benzylamine) coated particle, and a Poly(N-[3-(Dimethylamino) propyl] methacrylamide-co-[2-(methacryloyloxy)ethyl]dimethyl-(3-sulfopropyl) ammonium hydroxide, P (DMAPMA-co-SBMA) coated particle. A particle panel consistent with the present disclosure may comprise silica-coated particles, N-(3-Trimethoxysilylpropyl) diethylenetriamine coated particles, poly(N-(3-(dimethylamino) propyl) methacrylamide) (PDMAPMA)-coated particles, phosphate-sugar functionalized polystyrene particles, amine functionalized polystyrene particles, polystyrene carboxyl functionalized particles, ubiquitin functionalized polystyrene particles, dextran coated particles, or any combination thereof.

A particle panel consistent with the present disclosure may comprise a silica functionalized particle, an amine functionalized particle, a silicon alkoxide functionalized particle, a carboxylate functionalized particle, and a benzyl or phenyl functionalized particle. A particle panel consistent with the present disclosure may comprise a silica functionalized particle, an amine functionalized particle, a silicon alkoxide functionalized particle, a polystyrene functionalized particle, and a saccharide functionalized particle. A particle panel consistent with the present disclosure may comprise a silica functionalized particle, an N-(3-Trimethoxysilylpropyl) diethylenetriamine functionalized particle, a PDMAPMA functionalized particle, a dextran functionalized particle, and a polystyrene carboxyl functionalized particle. A particle panel consistent with the present disclosure may comprise 5 particles including a silica functionalized particle, an amine functionalized particle, a silicon alkoxide functionalized particle.

### Biomolecule Coronas

The present disclosure provides a variety of compositions, systems, and methods for collecting biomolecules on nanoparticles and microparticles (as well as other types of sensor elements such as polymer matrices, filters, rods, and extended surfaces). A particle may adsorb a plurality of biomolecules upon contact with a biological sample, thereby forming a biomolecule corona on the surfaces of the particles. The biomolecule corona may comprise proteins, lipids, nucleic acids, metabolites, saccharides, small molecules (e.g., sterols), and other biological species present in a sample. A biomolecule corona, as used herein, include all constituents adsorbed to a particle (e.g., proteins, lipids, nucleic acids, and other biomolecules).

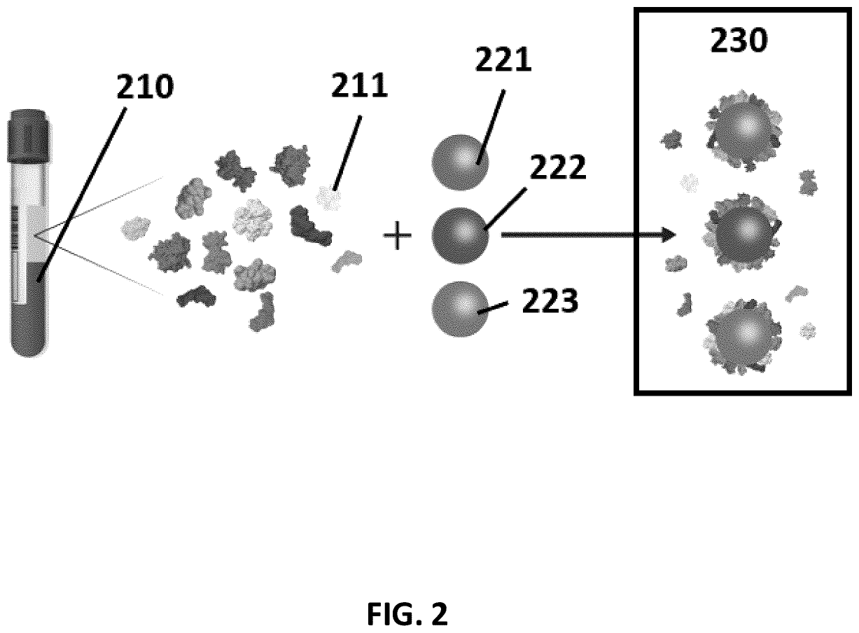

FIG. 2 provides a schematic overview of biomolecule formation, wherein a plurality of particles 221, 222, & 223 particles are contacted with a biological sample 210 comprising biomolecules molecules 211, and wherein each particle adsorbs a plurality of biomolecules from the biological sample to its surface 230. The different particles may be distinct particle types (depicted in the center of the figure, with the top, middle, and bottom spheres representing the three distinct particle types), such that each particle differs from the other particles by at least one physicochemical property. This difference in physicochemical properties can lead to the formation of different protein corona compositions on the particle surfaces.

The composition of the biomolecule corona may depend on a property of the particle.

In many cases, the composition of the biomolecule corona is strongly dependent on the surface of the particle. Characteristics such as particle surface material (e.g., ceramic, polymer, metal, metal oxide, graphite, silicon dioxide, etc.), surface texture (rough, smooth, grooved, etc.), surface functionalization (e.g., carboxylate functionalized, amine functionalized, small molecule (e.g., saccharide) functionalized, etc.), shape, curvature, and size can each independently serve as major determinants for biomolecule corona composition. In addition to surface features, the particle core composition, particle density, and particle surface area to mass ratio may each influence biomolecule corona composition. For example, two particles comprising the same surfaces and different cores may form different biomolecule coronas upon contact with the same sample.

Biomolecule corona formation may also be influenced by sample composition. For example, a first sample condition (e.g., low salinity) might favor the solubility of a particular analyte (e.g., an isoform of Bone Morphogenic Protein 1 (BMP1)), and thereby disfavor its binding in a biomolecule corona, while a second sample condition (e.g., high salinity) may diminish the solubility of the analyte, thereby driving its incorporation into a biomolecule corona.

Biomolecule corona composition may also depend on molecular level interactions between the biomolecules, themselves. An energetically favorable interaction between two biomolecules may promote their co-incorporation into a biomolecule corona. For example, if a first protein adsorbed to a particle comprises an affinity for a second protein in solution, the first protein may bind to a portion of the second protein, thereby driving its binding to the particle or to other proteins of the biomolecule corona of the particle. Analogously, a first biomolecule disposed within a biomolecule corona may comprise an energetically unfavorable interaction with a second biomolecule in a biological sample, thereby disfavoring its incorporation into a biomolecule corona. In part owing to these inter-biomolecule dependencies, biomolecule coronas provide sensitive platforms for directly and indirectly sensing biomolecules from a biological sample.

### Protein Analysis Methods

The particles and methods of use thereof disclosed herein can bind a large number of unique biomolecules (e.g., proteins) in a biological sample (e.g., a biofluid). For example, a particle disclosed herein can be incubated with a biological sample to form a protein corona comprising at least 5 unique proteins, at least 10 unique proteins, at least 15 unique proteins, at least 20 unique proteins, at least 25 unique proteins, at least 30 unique proteins, at least 40 unique proteins, at least 50 unique proteins, at least 60 unique proteins, at least 80 unique proteins, 100 unique proteins, at least 120 unique proteins, at least 140 unique proteins, at least 160 unique proteins, at least 180 unique proteins, at least 200 unique proteins, at least 220 unique proteins, at least 240 unique proteins, at least 260 unique proteins, at least 280 unique proteins, at least 300 unique proteins, at least 320 unique proteins, at least 340 unique proteins, at least 360 unique proteins, at least 380 unique proteins, at least 400 unique proteins, at least 420 unique proteins, at least 440 unique proteins, at least 460 unique proteins, at least 480 unique proteins, at least 500 unique proteins, at least 520 unique proteins, at least 540 unique proteins, at least 560 unique proteins, at least 580 unique proteins, at least 600 unique proteins, at least 620 unique proteins, at least 640 unique proteins, at least 660 unique proteins, at least 680 unique proteins, at least 700 unique proteins, at least 720 unique proteins, at least 740 unique proteins, at least 760 unique proteins, at least 780 unique proteins, at least 800 unique proteins, at least 820 unique proteins, at least 840 unique proteins, at least 860 unique proteins, at least 880 unique proteins, at least 900 unique proteins, at least 920 unique proteins, at least 940 unique proteins, at least 960 unique proteins, at least 980 unique proteins, at least 1000 unique proteins, at least 1100 unique proteins, at least 1200 unique proteins, at least 1300 unique proteins, at least 1400 unique proteins, at least 1500 unique proteins, at least 1600 unique proteins, at least 1800 unique proteins, at least 2000 unique proteins, from 100 to 2000 unique proteins, from 150 to 1500 unique proteins, from 200 to 1200 unique proteins, from 250 to 850 unique proteins, from 300 to 800 unique proteins, from 350 to 750 unique proteins, from 400 to 700 unique proteins, from 450 to 650 unique proteins, from 500 to 600 unique proteins, from 200 to 250 unique proteins, from 250 to 300 unique proteins, from 300 to 350 unique proteins, from 350 to 400 unique proteins, from 400 to 450 unique proteins, from 450 to 500 unique proteins, from 500 to 550 unique proteins, from 550 to 600 unique proteins, from 600 to 650 unique proteins, from 650 to 700 unique proteins, from 700 to 750 unique proteins, from 750 to 800 unique proteins, from 800 to 850 unique proteins, from 850 to 900 unique proteins, from 900 to 950 unique proteins, from 950 to 1000 unique proteins, or over 1000 unique proteins. In some cases, several different types of particles can be used, separately or in combination, to identify large numbers of proteins in a particular biological sample. In other words, particles can be multiplexed in order to bind and identify large numbers of proteins in a biological sample. Protein corona analysis may compress the dynamic range of the analysis compared to a protein analysis of the original sample.

The particle panels disclosed herein can be used to identify the number of distinct proteins disclosed herein, and/or any of the specific proteins disclosed herein, over a wide dynamic range. As used herein, a dynamic range may denote a log₁₀ value of a ratio of the highest and lowest abundance species of a specified type. Enriching or assaying species over a dynamic range may refer to the abundances of those species in the sample from which they were assayed or derived. For example, the particle panels disclosed herein comprising distinct particle types, can enrich for proteins in a sample, which can be identified using a workflow, for example, as described herein and disclosed in U.S. Pat. No. 11,428,688, which is incorporated by reference herein in its entirety, over the entire dynamic range at which proteins are present in a sample (e.g., a plasma sample). In some cases, a particle panel including any number of distinct particle types disclosed herein, enriches and identifies proteins over a dynamic range of at least 2. In some cases, a particle panel including any number of distinct particle types disclosed herein, enriches and identifies proteins over a dynamic range of at least 3. In some cases, a particle panel including any number of distinct particle types disclosed herein, enriches and identifies proteins over a dynamic range of at least 4. In some cases, a particle panel including any number of distinct particle types disclosed herein, enriches and identifies proteins over a dynamic range of at least 5. In some cases, a particle panel including any number of distinct particle types disclosed herein, enriches and identifies proteins over a dynamic range of at least 6. In some cases, a particle panel including any number of distinct particle types disclosed herein, enriches and identifies proteins over a dynamic range of at least 7. In some cases, a particle panel including any number of distinct particle types disclosed herein, enriches and identifies proteins over a dynamic range of at least 8. In some cases, a particle panel including any number of distinct particle types disclosed herein, enriches and identifies proteins over a dynamic range of at least 9. In some cases, a particle panel including any number of distinct particle types disclosed herein, enriches and identifies proteins over a dynamic range of at least 10. In some cases, a particle panel including any number of distinct particle types disclosed herein, enriches and identifies proteins over a dynamic range of at least 11. In some cases, a particle panel including any number of distinct particle types disclosed herein, enriches and identifies proteins over a dynamic range of at least 12. In some cases, a particle panel including any number of distinct particle types disclosed herein, enriches and identifies proteins over a dynamic range of at least 13. In some cases, a particle panel including any number of distinct particle types disclosed herein, enriches and identifies proteins over a dynamic range of at least 14. In some cases, a particle panel including any number of distinct particle types disclosed herein, enriches and identifies proteins over a dynamic range of at least 15. In some cases, a particle panel including any number of distinct particle types disclosed herein, enriches and identifies proteins over a dynamic range of at least 20. In some cases, a particle panel including any number of distinct particle types disclosed herein, enriches and identifies proteins over a dynamic range of from 2 to 100. In some cases, a particle panel including any number of distinct particle types disclosed herein, enriches and identifies proteins over a dynamic range of from 2 to 20. In some cases, a particle panel including any number of distinct particle types disclosed herein, enriches and identifies proteins over a dynamic range of from 2 to 10. In some cases, a particle panel including any number of distinct particle types disclosed herein, enriches and identifies proteins over a dynamic range of from 2 to 5. In some cases, a particle panel including any number of distinct particle types disclosed herein, enriches and identifies proteins over a dynamic range of from 5 to 10.

Some aspects of the devices and methods disclosed herein may solve some of these challenges by “compressing” the dynamic range of protein species in a sample. Some aspects of the devices and methods disclosed herein operate based on non-specific binding of proteins to nanoparticle surfaces to form protein coronas. Without requiring a presence of a specific entity that is configured for binding to a singular specific protein (e.g., as in immunoassays), the non-specific binding can result in a dynamic range compression of proteins bound to the nanoparticle surfaces while capturing a wide variety of proteins. In other words, the relative abundance of proteins in the sample can be modified on the nanoparticle surfaces, such that the rare proteins are relatively more abundant, and the highly abundant proteins are relatively less abundant compared to the original sample. The proteins can then be separated from the sample and analyzed, for example, with mass spectrometry. The compressed dynamic range can allow rare proteins to comprise a higher fraction of ionic species, thereby allowing higher probability for detecting those rare proteins in a MS experiment. Though the above example is described in terms of proteins, other biomolecule classes (e.g., lipids, sugars, etc.) can be similarly targeted. Other aspects of the methods and devices disclosed herein include controlled automation of the workflow that increases speed/throughput and accuracy/reliability.

Due to individual differences in biology between humans, thousands of proteins can have varying levels in plasma samples between two individuals. Therefore, samples from hundreds or thousands of individuals may be experimented with to identify meaningful and systematic signals that have clinical relevance. Thus, some aspects of the present disclosure provides systems, compositions, and methods, that provide higher depth (e.g., increased number of detected biomolecules and increased dynamic range) and throughput, which can identify more subtle minutiae present in biological signatures of complex biological samples. By increasing depth, more obscure biological signatures (e.g., those associated with very rare proteins) can become more visible. By increasing throughput, spurious variations in data (e.g., associated with known and unknown sources of error, i.e., standard deviation/error) can be mitigated by improving certainty.

Biomolecules collected on a particle may be subjected to further analysis. A method may comprise collecting a biomolecule corona or a subset of biomolecules from a biomolecule corona. The collected biomolecule corona or the collected subset of biomolecules from the biomolecule corona may be subjected to further particle-based analysis (e.g., particle adsorption). The collected biomolecule corona or the collected subset of biomolecules from the biomolecule corona may be purified or fractionated (e.g., by a chromatographic method). The collected biomolecule corona or the collected subset of biomolecules from the biomolecule corona may be analyzed (e.g., by mass spectrometry).

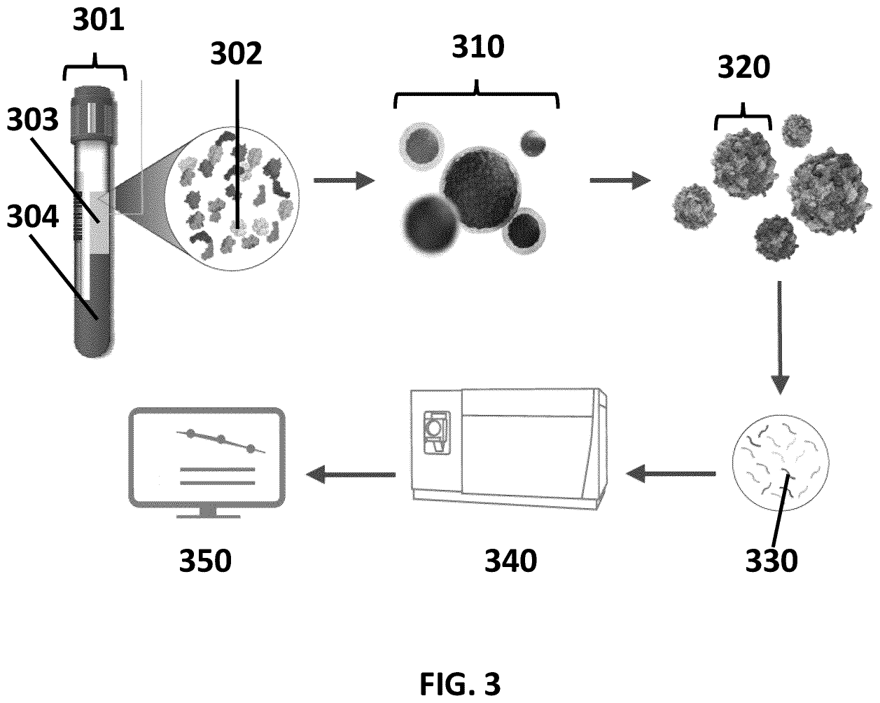

FIG. 3 provides an example of a particle-based biomolecule corona (e.g., protein corona) assay consistent with the present disclosure. A biological sample (e.g., human plasma) 301 comprising a plurality of biomolecules 302 may be contacted to a plurality of particles 310. The sample may be treated, diluted, or split into a plurality of fractions 303 and 304 prior to analysis. For example, a whole blood sample may be fractionated into plasma and erythrocyte portions. Upon contact with the particles, a subset or the entirety of the plurality of biomolecules may adsorb to the particles, thereby forming biomolecule coronas 320 bound to the surfaces of the particles. Unbound biomolecules may be separated from the biomolecule coronas (e.g., through wash steps). The biomolecule coronas, or subsets thereof, may be collected from the particles. Alternatively, biomolecules of the biomolecule coronas may be fragmented or chemically treated while bound to the particles. In some assays, biomolecules (e.g., proteins) are fragmented (e.g., digested) while disposed in the biomolecule coronas to yield biomolecule (e.g., peptide) fragments 330. Biomolecules (or their chemically treated or fragmented derivatives) may be analyzed 340, for example by mass spectrometry, to yield data 350 representative of biomolecules 302 from the biological sample 301. The data may be analyzed to identify a biological state of the biological sample.

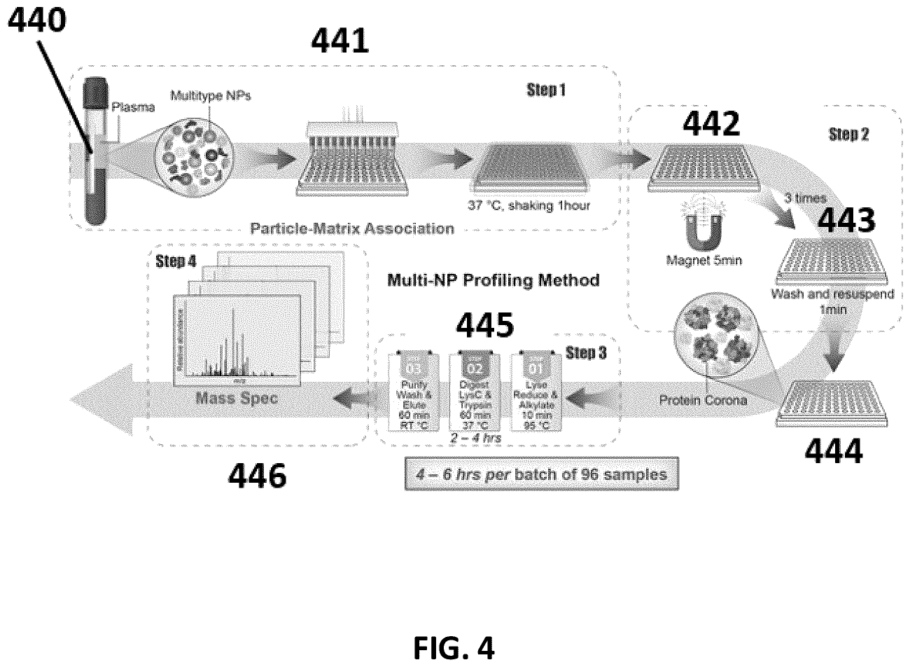

FIG. 4 illustrates an example of a biomolecule corona (e.g., protein corona) analysis workflow consistent with the present disclosure which includes: particle incubation with a biological sample 440 (e.g., plasma), thereby adsorbing biomolecules from the plasma sample to the particles to form biomolecule coronas; partitioning 441 of the particle-plasma sample mixture into a plurality of wells on a 96 well plate; particle collection 442 (e.g., with a magnet); a wash step or plurality of wash steps 443 to remove analytes not adsorbed to the particles; 444 resuspension of the particles and the biomolecules adsorbed thereto; optionally, biomolecule corona digestion or chemical treatment 445 (e.g., protein reduction and digestion); and analysis of the biomolecule coronas or of biomolecules derived therefrom 446 (e.g., by liquid chromatography-mass spectrometry (LC-MS) analysis). While this example provides parallel analyses across 96 well plate wells, a method may comprise a single sample volume or a plurality of sample volumes ranging from two to hundreds of thousands of sample volumes. Furthermore, while this example provides contacting a sample with particles prior to partitioning, a method may alternatively comprise partitioning a sample (e.g., into separate wells of a well plate) prior to contacting with particles. In some cases, sample may be added to partitions comprising particles. For example, a well plate may be provided with particles, buffer, and reagents in dry form, such that a method of use may comprise adding solution to the wells to resuspend the particles and dissolve the buffer and reagents, and then adding sample to the wells.

Protein corona analysis may comprise an automated component. For example, an automated instrument may contact a sample with a particle or particle panel, identify proteins on the particle or particle panel (e.g., digest the proteins on the particle or particle panel and perform mass spectrometric analysis), and generate data for identifying a specific biomolecule or a biological state of a sample. The automated instrument may divide a sample into a plurality of volumes, and perform analysis on each volume. The automated instrument may analyze multiple separate samples, for example by disposing multiple samples within multiple wells in a well plate and performing parallel analysis on each sample. In some aspects, the present disclosure provides an automated apparatus to generate a subset of biomolecules from a biological sample or identify proteins in a biological sample, using an automated apparatus disclosed in U.S. Pat. No. 11,630,112 and incorporated herein by reference in its entirety, and other automated systems.

The particles disclosed herein can be used to identify a number of proteins, peptides, protein groups, or protein classes using a protein analysis workflow described herein (e.g., a protein corona analysis workflow). Protein corona analysis may comprise contacting a sample to distinct particle types (e.g., a particle panel), forming biomolecule corona on the distinct particle types, and identifying the biomolecules in the biomolecule corona (e.g., by mass spectrometry). Feature intensities, as disclosed herein, refers to the intensity of a discrete spike (“feature”) seen on a plot of mass to charge ratio versus intensity from a mass spectrometry run of a sample. These features can correspond to variably ionized fragments of peptides and/or proteins. Using the data analysis methods described herein, feature intensities can be sorted into protein groups. Protein groups refer to two or more proteins that are identified by a shared peptide sequence. Alternatively, a protein group can refer to one protein that is identified using a unique identifying sequence. For example, if in a sample, a peptide sequence is assayed that is shared between two proteins (Protein 1: XYZZX and Protein 2: XYZYZ), a protein group could be the “XYZ protein group” having two members (protein 1 and protein 2). Alternatively, if the peptide sequence is unique to a single protein (Protein 1), a protein group could be the “ZZX” protein group having one member (Protein 1). Each protein group can be supported by more than one peptide sequence. Protein detected or identified according to the instant disclosure can refer to a distinct protein detected in the sample (e.g., distinct relative other proteins detected using mass spectrometry). Thus, analysis of proteins present in distinct coronas corresponding to the distinct particle types in a particle panel yields a high number of feature intensities. This number decreases as feature intensities are processed into distinct peptides, further decreases as distinct peptides are processed into distinct proteins, and further decreases as peptides are grouped into protein groups (two or more proteins that share a distinct peptide sequence).

The methods disclosed herein include isolating one or more particle types from a sample or from more than one sample (e.g., a biological sample or a serially interrogated sample). The particle types can be rapidly isolated or separated from the sample using a magnet. Moreover, multiple samples that are spatially isolated can be processed in parallel. Thus, the methods disclosed herein provide for isolating or separating a particle type from unbound protein in a sample. A particle type may be separated by a variety of means, including but not limited to magnetic separation, centrifugation, filtration, or gravitational separation. Particle panels may be incubated with a plurality of spatially isolated samples, wherein each spatially isolated sample is in a well in a well plate (e.g., a 96-well plate). After incubation, the particle types in each of the wells of the well plate can be separated from unbound protein present in the spatially isolated samples by placing the entire plate on a magnet. This simultaneously pulls down the superparamagnetic particles in the particle panel. The supernatant in each sample can be removed to remove the unbound protein. These steps (incubate, pull down) can be repeated to effectively wash the particles, thus removing residual background unbound protein that may be present in a sample. This is one example, but one of skill in the art could envision numerous other scenarios in which superparamagnetic particles are rapidly isolated from one or more than one spatially isolated samples at the same time.

The methods and compositions of the present disclosure provide identification and measurement of particular proteins in the biological samples by processing of the proteomic data via digestion of coronas formed on the surface of particles. Examples of proteins that can be identified and measured include highly abundant proteins, proteins of medium abundance, and low-abundance proteins. A low abundance protein may be present in a sample at concentrations at or below about 10 ng/mL. A high abundance protein may be present in a sample at concentrations at or above about 10 μg/mL. A protein of moderate abundance may be present in a sample at concentrations between about 10 ng/ml and about 10 μg/mL. Examples of proteins that are highly abundant proteins include albumin, IgG, and the top 14 proteins in abundance that contribute 95% of the analyte mass in plasma. Additionally, any proteins that may be purified using a conventional depletion column may be directly detected in a sample using the particle panels disclosed herein. Examples of proteins may be any protein listed in published databases such as Keshishian et al. (Mol Cell Proteomics. 2015 September; 14 (9): 2375-93. doi: 10.1074/mcp.M114.046813. Epub 2015 Feb. 27.), Farr et al. (J Proteome Res. 2014 Jan. 3; 13 (1): 60-75. doi: 10.1021/pr4010037. Epub 2013 Dec. 6.), or Pernemalm et al. (Expert Rev Proteomics. 2014 August; 11 (4): 431-48. doi: 10.1586/14789450.2014.901157. Epub 2014 Mar. 24.).

The methods and compositions disclosed herein may also elucidate protein classes or interactions of the protein classes. A protein class may comprise a set of proteins that share a common function (e.g., amine oxidases or proteins involved in angiogenesis); proteins that share common physiological, cellular, or subcellular localization (e.g., peroxisomal proteins or membrane proteins); proteins that share a common cofactor (e.g., heme or flavin proteins); proteins that correspond to a particular biological state (e.g., hypoxia related proteins); proteins containing a particular structural motif (e.g., a cupin fold); or proteins bearing a post-translational modification (e.g., ubiquitinated or citrullinated proteins). A protein class may contain at least 2 proteins, 5 proteins, 10 proteins, 20 proteins, 40 proteins, 60 proteins, 80 proteins, 100 proteins, 150 proteins, 200 proteins, or more.

The proteomic data of the biological sample can be identified, measured, and quantified using a number of different analytical techniques. For example, proteomic data can be generated using SDS-PAGE or any gel-based separation technique. Peptides and proteins can also be identified, measured, and quantified using an immunoassay, such as ELISA. Alternatively, proteomic data can be identified, measured, and quantified using mass spectrometry, high performance liquid chromatography, LC-MS/MS, Edman Degradation, immunoaffinity techniques, methods disclosed in U.S. Pat. No. 10,866,242, EP3548652, WO2019083856, WO2019133892, each of which is incorporated by reference herein in their entirety, and other protein separation techniques.

An assay may comprise protein collection of particles, protein digestion, and mass spectrometric analysis (e.g., MS, LC-MS, LC-MS/MS). The digestion may comprise chemical digestion, such as by cyanogen bromide or 2-Nitro-5-thiocyanatobenzoic acid (NTCB). The digestion may comprise enzymatic digestion, such as by trypsin or pepsin. The digestion may comprise enzymatic digestion by a plurality of proteases. The digestion may comprise a protease selected from among the group consisting of trypsin, chymotrypsin, endoproteinase Glu C, endoproteinase Lys C, elastase, subtilisin, proteinase K, thrombin, factor X, endoproteinase Arg C, papain, endoproteinase AspN, thermolysin, pepsin, aspartyl protease, cathepsin D, zinc mealloprotease, glycoprotein endopeptidase, aminopeptidase, prenyl protease, caspase, kex2 endoprotease, or any combination thereof. The digestion may cleave peptides at random positions. The digestion may cleave peptides at a specific position (e.g., at methionines) or sequence (e.g., glutamate-histidine-glutamate). The digestion may enable similar proteins to be distinguished. For example, an assay may resolve 8 distinct proteins as a single protein group with a first digestion method, and as 8 separate proteins with distinct signals with a second digestion method. The digestion may generate an average peptide fragment length of 8 to 15 amino acids. The digestion may generate an average peptide fragment length of 12 to 18 amino acids. The digestion may generate an average peptide fragment length of 15 to 25 amino acids. The digestion may generate an average peptide fragment length of 20 to 30 amino acids. The digestion may generate an average peptide fragment length of 30 to 50 amino acids.

An assay may rapidly generate and analyze proteomic data. Beginning with an input biological sample (e.g., a buccal or nasal smear, plasma, or tissue), an assay of the present disclosure may generate and analyze proteomic data in less than 7 hours. Beginning with an input biological sample, an assay of the present disclosure may generate and analyze proteomic data in 5-7 hours. Beginning with an input biological sample, an assay of the present disclosure may generate and analyze proteomic data in less than 5 hours. Beginning with an input biological sample, an assay of the present disclosure may generate and analyze proteomic data in 3-5 hours. Beginning with an input biological sample, an assay of the present disclosure may generate and analyze proteomic data in 2-4 hours. Beginning with an input biological sample, an assay of the present disclosure may generate and analyze proteomic data in 2-3 hours. Beginning with an input biological sample, an assay of the present disclosure may generate and analyze proteomic data in less than 3 hours. Beginning with an input biological sample, an assay of the present disclosure may generate and analyze proteomic data in less than 2 hours. The analyzing may comprise identifying a protein group. The analyzing may comprise identifying a protein class. The analyzing may comprise quantifying an abundance of a biomolecule, a peptide, a protein, protein group, or a protein class. The analyzing may comprise identifying a ratio of abundances of two biomolecules, peptides, proteins, protein groups, or protein classes. The analyzing may comprise identifying a biological state.

### Dynamic Range

The biomolecule corona analysis methods described herein may comprise assaying biomolecules in a sample of the present disclosure across a wide dynamic range. The dynamic range of biomolecules assayed in a sample may be a range of measured signals of biomolecule abundances as measured by an assay method (e.g., mass spectrometry, chromatography, gel electrophoresis, spectroscopy, or immunoassays) for the biomolecules contained within a sample. For example, an assay capable of detecting proteins across a wide dynamic range may be capable of detecting proteins of very low abundance to proteins of very high abundance. The dynamic range of an assay may be directly related to the slope of assay signal intensity as a function of biomolecule abundance. For example, an assay with a low dynamic range may have a low (but positive) slope of the assay signal intensity as a function of biomolecule abundance, e.g., the ratio of the signal detected for a high abundance biomolecule to the ratio of the signal detected for a low abundance biomolecule may be lower for an assay with a low dynamic range than an assay with a high dynamic range. In specific cases, dynamic range may refer to the dynamic range of proteins within a sample or assaying method.

The biomolecule corona analysis methods described herein may compress the dynamic range of an assay. The dynamic range of an assay may be compressed relative to another assay if the slope of the assay signal intensity as a function of biomolecule abundance is lower than that of the other assay. For example, a plasma sample assayed using protein corona analysis with mass spectrometry may have a compressed dynamic range compared to a plasma sample assayed using mass spectrometry alone, directly on the sample or compared to provided abundance values for plasma proteins in databases (e.g., the database provided in Keshishian et al., Mol. Cell Proteomics 14, 2375-2393 (2015), also referred to herein as the “Carr database”). The compressed dynamic range may enable the detection of more low abundance biomolecules in a biological sample using biomolecule corona analysis with mass spectrometry than using mass spectrometry alone.

In some embodiments, the dynamic range of a proteomic analysis assay may be the ratio of the signal produced by highest abundance proteins (e.g., the highest 10% of proteins by abundance) to the signal produced by the lowest abundance proteins (e.g., the lowest 10% of proteins by abundance). Compressing the dynamic range of a proteomic analysis may comprise decreasing the ratio of the signal produced by the highest abundance proteins to the signal produced by the lowest abundance proteins for a first proteomic analysis assay relative to that of a second proteomic analysis assay. The protein corona analysis assays disclosed herein, as well as those disclosed in U.S. Pat. No. 11,428,688, which is incorporated by reference herein in its entirety, may compress the dynamic range relative to the dynamic range of a total protein analysis method (e.g., mass spectrometry, gel electrophoresis, or liquid chromatography).

Provided herein are several methods for compressing the dynamic range of a biomolecular analysis assay to facilitate the detection of low abundance biomolecules relative to high abundance biomolecules. For example, a particle type of the present disclosure can be used to serially interrogate a sample. Upon incubation of the particle type in the sample, a biomolecule corona comprising forms on the surface of the particle type. If biomolecules are directly detected in the sample without the use of the particle types, for example by direct mass spectrometric analysis of the sample, the dynamic range may span a wider range of concentrations, or more orders of magnitude, than if the biomolecules are directed on the surface of the particle type. Thus, using the particle types disclosed herein may be used to compress the dynamic range of biomolecules in a sample. Without being limited by theory, this effect may be observed due to more capture of higher affinity, lower abundance biomolecules in the biomolecule corona of the particle type and less capture of lower affinity, higher abundance biomolecules in the biomolecule corona of the particle type.

A dynamic range of a proteomic analysis assay may be the slope of a plot of a protein signal measured by the proteomic analysis assay as a function of total abundance of the protein in the sample. Compressing the dynamic range may comprise decreasing the slope of the plot of a protein signal measured by a proteomic analysis assay as a function of total abundance of the protein in the sample relative to the slope of the plot of a protein signal measured by a second proteomic analysis assay as a function of total abundance of the protein in the sample. The protein corona analysis assays disclosed herein may compress the dynamic range relative to the dynamic range of a total protein analysis method (e.g., mass spectrometry, gel electrophoresis, or liquid chromatography).

### Kits

Provided herein are kits comprising compositions of the present disclosure that may be used to perform the methods of the present disclosure. A kit may comprise one or more particle types to interrogate a sample to identify a biological state of a sample. In some cases, a kit may comprise a particle type provided in TABLE 1. A kit may comprise a reagent for functionalizing a particle (e.g., a reagent for tethering a small molecule functionalization to a particle surface). The kit may be pre-packaged in discrete aliquots. In some cases, the kit can comprise a plurality of different particle types that can be used to interrogate a sample. The plurality of particle types can be pre-packaged where each particle type of the plurality is packaged separately. Alternately, the plurality of particle types can be packaged together to contain combination of particle types in a single package. A particle may be provided in dried (e.g., lyophilized) form, or may be provided in a suspension or solution. The particles may be provided in a well plate. For example, a kit may contain an 8 well plate, an 8-384 well plate with particles provided (e.g., sealed) within the wells. For example, a well plate may comprise at least 8, at least 16, at least 24, at least 32, at least 40, at least 48, at least 56, at least 64, at least 72, at least 80, at least 88, at least 96, at least 104, at least 112, at least 120, at least 128, at least 136, at least 144, at least 152, at least 160, at least 168, at least 176, at least 184, at least 192, at least 200, at least 208, at least 216, at least 224, at least 232, at least 240, at least 248, at least 256, at least 264, at least 272, at least 280, at least 288, at least 296, at least 304, at least 312, at least 320, at least 328, at least 336, at least 344, at least 352, at least 360, at least 368, at least 376, at least 384, at least 392, at least 400 wells comprising particles. Two wells in such a well plate may contain different particles or different concentrations of particles. Two wells may comprise different buffers or chemical conditions. For example, a well plate may be provided with different particles in each row of wells and different buffers in each column of rows. A well may be sealed by a removable covering. For example, a kit may comprise a well plate comprising a plastic slip covering a plurality of wells. A well may be sealed by a pierceable covering. For example, a well may be covered by a septum that a needle can pierce to facilitate sample movement into and out of the well.

### Samples

The present disclosure provides a range of samples that can be assayed using the particles and the methods provided herein. A sample may be a biological sample (e.g., a sample derived from a living organism). A sample may comprise a cell or be cell-free. A sample may comprise a biofluid, such as blood, serum, plasma, urine, or cerebrospinal fluid (CSF). Samples consistent with the present disclosure include biological samples from a subject. The subject may be a human or a non-human animal. The biological samples can contain a plurality of proteins or proteomic data, which may be analyzed after adsorption of proteins to the surface of the various sensor element (e.g., particle) types in a panel and subsequent digestion of protein coronas. Proteomic data can comprise nucleic acids, peptides, or proteins. A biofluid may be a fluidized solid, for example a tissue homogenate, or a fluid extracted from a biological sample. A biological sample may be, for example, a tissue sample or a fine needle aspiration (FNA) sample. A biological sample may be a cell culture sample. For example, a biofluid may be a fluidized cell culture extract.

A wide range of samples are compatible for use within the methods and compositions of the present disclosure. The biological sample may comprise plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof. The biological sample may comprise multiple biological samples (e.g., pooled plasma from multiple subjects, or multiple tissue samples from a single subject). The biological sample may comprise a single type of biofluid or biomaterial from a single source.

The biological sample may be diluted or pre-treated. The biological sample may undergo depletion (e.g., the biological sample comprises serum) prior to or following contact with a particle or plurality of particles. The biological sample may also undergo physical (e.g., homogenization or sonication) or chemical treatment prior to or following contact with a particle or plurality of particles. The biological sample may be diluted prior to or following contact with a particle or plurality of particles. The dilution medium may comprise buffer or salts, or be purified water (e.g., distilled water). Different partitions of a biological sample may undergo different degrees of dilution. A biological sample or a portion thereof may undergo a 1.1-fold, 1.2-fold, 1.3-fold, 1.4-fold, 1.5-fold, 2-fold, 3-fold, 4-fold, 5-fold, 6-fold, 8-fold, 10-fold, 12-fold, 15-fold, 20-fold, 30-fold, 40-fold, 50-fold, 75-fold, 100-fold, 200-fold, 500-fold, or 1000-fold dilution.

The compositions and methods of the present disclosure can be used to measure, detect, and identify specific proteins from biological samples. Examples of proteins that can be identified and measured include highly abundant proteins, proteins of medium abundance, and low-abundance proteins. For example, a composition or method may identify at least 1, at least 2, at least 3, at least 4, at least 5, at least 6, at least 7, at least 8, at least 10, at least 12, at least 15, at least 18, at least 20, at least 25, at least 30, at least 35, at least 40, or at least 50 human plasma proteins from the group consisting of albumin, immunoglobulin G (IgG), lysozyme, carcino embryonic antigen (CEA), receptor tyrosine-protein kinase erbB-2 (HER-2/neu), bladder tumor antigen, thyroglobulin, alpha-fetoprotein, prostate specific antigen (PSA), mucin 16 (CA125), carbohydrate antigen 19-9 (CA19.9), carcinoma antigen 15-3 (CA15.3), leptin, prolactin, osteopontin, insulin-like growth factor 2 (IGF-II), 4F2 cell-surface antigen heavy chain (CD98), fascin, sPigR, 14-3-3 eta, troponin I, B-type natriuretic peptide, breast cancer type 1 susceptibility protein (BRCA1), c-Myc proto-oncogene protein (c-Myc), interleukin-6 (IL-6), fibrinogen, epidermal growth factor receptor (EGFR), gastrin, PH, granulocyte colony-stimulating factor (G CSF), desmin, enolase 1 (NSE), folice-stimulating hormone (FSH), vascular endothelial growth factor (VEGF), P21, Proliferating cell nuclear antigen (PCNA), calcitonin, pathogenesis-related proteins (PR), luteinizing hormone (LH), somatostatin S100, insulin. alpha-prolactin, adrenocorticotropic hormone (ACTH), B-cell lymphoma 2 (Bcl 2), estrogen receptor alpha (ER alpha), antigen k (Ki-67), tumor protein (p53), cathepsin D, beta catenin, von Willebrand factor (VWF), CD15, k-ras, caspase 3, ENTH domain-containing protein (EPN), CD10, FAS, breast cancer type 2 susceptibility protein (BRCA2), CD30L, CD30, CGA, CRP, prothrombin, CD44, APEX, transferrin, GM-CSF, E-cadherin, interleukin-2 (IL-2), Bax, IFN-gamma, beta-2-MG, tumor necrosis factor alpha (TNF alpha), cluster of differentiation 340, trypsin, cyclin D1, MG B, XBP-1, HG-1, YKL-40, S-gamma, ceruloplasmin, NESP-55, netrin-1, geminin, GADD45A, CDK-6, CCL21, breast cancer metastasis suppressor 1 (BrMS1), 17betaHDI, platelet-derived growth factor receptor A (PDGRFA), P300/CBP-associated factor (Pcaf), chemokine ligand 5 (CCL5), matrix metalloproteinase-3 (MMP3), claudin-4, and claudin-3

### Biological States

The compositions and methods disclosed herein can be used to identify various biological states in a particular biological sample. For example, a biological state can refer to an elevated or low level of a particular protein or a set of proteins. In other examples, a biological state can refer to identification of a disease, such as cancer. The particles and methods of us thereof can be used to distinguish between two biological states. The two biological states may be related diseases states (e.g., two HRAS mutant colon cancers or different stages of a type of a cancer). The two biological states may be different phases of a disease, such as pre-Alzheimer's and mild Alzheimer's. The two biological states may be distinguished with a high degree of accuracy (e.g., the percentage of accurately identified biological states among a population of samples). For example, the compositions and methods of the present disclosure may distinguish two biological states with at least 60% accuracy, at least 70% accuracy, at least 75% accuracy at least 80% accuracy, at least 85% accuracy, at least 90% accuracy, at least 95% accuracy, at least 98% accuracy, or at least 99% accuracy. The two biological states may be distinguished with a high degree of specificity (e.g., the rate at which negative results are correctly identified among a population of samples). For example, the compositions and methods of the present disclosure may distinguish two biological states with at least 60% specificity, at least 70% specificity, at least 75% specificity at least 80% specificity, at least 85% specificity, at least 90% specificity, at least 95% specificity, at least 98% specificity, or at least 99% specificity.

The methods, compositions, and systems described herein can be used to determine a disease state, and/or prognose or diagnose a disease or disorder. The diseases or disorders contemplated include, but are not limited to, for example, cancer, cardiovascular disease, endocrine disease, inflammatory disease, a neurological disease and the like.

The methods, compositions, and systems described herein can be used to determine, prognose, and/or diagnose a cancer disease state. The term “cancer” is meant to encompass any cancer, neoplastic and preneoplastic disease that is characterized by abnormal growth of cells, including tumors and benign growths. Cancer may, for example, be lung cancer, pancreatic cancer, or skin cancer. In many cases, the methods, compositions and systems described herein are not only able to diagnose cancer (e.g. determine if a subject (a) does not have cancer, (b) is in a pre-cancer development stage, (c) is in early stage of cancer, (d) is in a late stage of cancer) but are able to determine the type of cancer.

The methods, compositions, and systems of the present disclosure can additionally be used to detect other cancers, such as acute lymphoblastic leukemia (ALL); acute myeloid leukemia (AML); cancer in adolescents; adrenocortical carcinoma; childhood adrenocortical carcinoma; unusual cancers of childhood; AIDS-related cancers; kaposi sarcoma (soft tissue sarcoma); AIDS-related lymphoma (lymphoma); primary cns lymphoma (lymphoma); anal cancer; appendix cancer-see gastrointestinal carcinoid tumors; astrocytomas, childhood (brain cancer); atypical teratoid/rhabdoid tumor, childhood, central nervous system (brain cancer); basal cell carcinoma of the skin-see skin cancer; bile duct cancer; bladder cancer; childhood bladder cancer; bone cancer (includes ewing sarcoma and osteosarcoma and malignant fibrous histiocytoma); brain tumors; breast cancer; childhood breast cancer; bronchial tumors, childhood; burkitt lymphoma-see non-hodgkin lymphoma; carcinoid tumor (gastrointestinal); childhood carcinoid tumors; carcinoma of unknown primary; childhood carcinoma of unknown primary; cardiac (heart) tumors, childhood; central nervous system; atypical teratoid/rhabdoid tumor, childhood (brain cancer); embryonal tumors, childhood (brain cancer); germ cell tumor, childhood (brain cancer); primary cns lymphoma; cervical cancer; childhood cervical cancer; childhood cancers; cancers of childhood, unusual; cholangiocarcinoma-see bile duct cancer; chordoma, childhood; chronic lymphocytic leukemia (CLL); chronic myelogenous leukemia (CML); chronic myeloproliferative neoplasms; colorectal cancer; childhood colorectal cancer; craniopharyngioma, childhood (brain cancer); cutaneous t-cell lymphoma-see lymphoma (mycosis fungoides and sézary syndrome); ductal carcinoma in situ (DCIS)-see breast cancer; embryonal tumors, central nervous system, childhood (brain cancer); endometrial cancer (uterine cancer); ependymoma, childhood (brain cancer); esophageal cancer; childhood esophageal cancer; esthesioneuroblastoma (head and neck cancer); ewing sarcoma (bone cancer); extracranial germ cell tumor, childhood; extragonadal germ cell tumor; eye cancer; childhood intraocular melanoma; intraocular melanoma; retinoblastoma; fallopian tube cancer; fibrous histiocytoma of bone, malignant, and osteosarcoma; gallbladder cancer; gastric (stomach) cancer; childhood gastric (stomach) cancer; gastrointestinal carcinoid tumor; gastrointestinal stromal tumors (GIST) (soft tissue sarcoma); childhood gastrointestinal stromal tumors; germ cell tumors; childhood central nervous system germ cell tumors (brain cancer); childhood extracranial germ cell tumors; extragonadal germ cell tumors; ovarian germ cell tumors; testicular cancer; gestational trophoblastic disease; hairy cell leukemia; head and neck cancer; heart tumors, childhood; hepatocellular (liver) cancer; histiocytosis, langerhans cell; hodgkin lymphoma; hypopharyngeal cancer (head and neck cancer); intraocular melanoma; childhood intraocular melanoma; islet cell tumors, pancreatic neuroendocrine tumors; kaposi sarcoma (soft tissue sarcoma); kidney (renal cell) cancer; langerhans cell histiocytosis; laryngeal cancer (head and neck cancer); leukemia; lip and oral cavity cancer (head and neck cancer); liver cancer; lung cancer (non-small cell and small cell); childhood lung cancer; lymphoma; male breast cancer; malignant fibrous histiocytoma of bone and osteosarcoma; melanoma; childhood melanoma; melanoma, intraocular (eye); childhood intraocular melanoma; merkel cell carcinoma (skin cancer); mesothelioma, malignant; childhood mesothelioma; metastatic cancer; metastatic squamous neck cancer with occult primary (head and neck cancer); midline tract carcinoma with nut gene changes; mouth cancer (head and neck cancer); multiple endocrine neoplasia syndromes; multiple myeloma/plasma cell neoplasms; mycosis fungoides (lymphoma); myelodysplastic syndromes, myelodysplastic/myeloproliferative neoplasms; myelogenous leukemia, chronic (cml); myeloid leukemia, acute (aml); myeloproliferative neoplasms, chronic; nasal cavity and paranasal sinus cancer (head and neck cancer); nasopharyngeal cancer (head and neck cancer); neuroblastoma; non-hodgkin lymphoma; non-small cell lung cancer; oral cancer, lip and oral cavity cancer and oropharyngeal cancer (head and neck cancer); osteosarcoma and malignant fibrous histiocytoma of bone; ovarian cancer; childhood ovarian cancer; pancreatic cancer; childhood pancreatic cancer; pancreatic neuroendocrine tumors (islet cell tumors); papillomatosis (childhood laryngeal); paraganglioma; childhood paraganglioma; paranasal sinus and nasal cavity cancer (head and neck cancer); parathyroid cancer; penile cancer; pharyngeal cancer (head and neck cancer); pheochromocytoma; childhood pheochromocytoma; pituitary tumor; plasma cell neoplasm/multiple myeloma; pleuropulmonary blastoma; pregnancy and breast cancer; primary central nervous system (CNS) lymphoma; primary peritoneal cancer; prostate cancer; rectal cancer; recurrent cancer; renal cell (kidney) cancer; retinoblastoma; rhabdomyosarcoma, childhood (soft tissue sarcoma); salivary gland cancer (head and neck cancer); sarcoma; childhood rhabdomyosarcoma (soft tissue sarcoma); childhood vascular tumors (soft tissue sarcoma); ewing sarcoma (bone cancer); kaposi sarcoma (soft tissue sarcoma); osteosarcoma (bone cancer); soft tissue sarcoma; uterine sarcoma; sézary syndrome (lymphoma); skin cancer; childhood skin cancer; small cell lung cancer; small intestine cancer; soft tissue sarcoma; squamous cell carcinoma of the skin-see skin cancer; squamous neck cancer with occult primary, metastatic (head and neck cancer); stomach (gastric) cancer; childhood stomach (gastric) cancer; t-cell lymphoma, cutaneous-see lymphoma (mycosis fungoides and sèzary syndrome); testicular cancer; childhood testicular cancer; throat cancer (head and neck cancer); nasopharyngeal cancer; oropharyngeal cancer; hypopharyngeal cancer; thymoma and thymic carcinoma; thyroid cancer; transitional cell cancer of the renal pelvis and ureter (kidney (renal cell) cancer); carcinoma of unknown primary; childhood cancer of unknown primary; unusual cancers of childhood; ureter and renal pelvis, transitional cell cancer (kidney (renal cell) cancer; urethral cancer; uterine cancer, endometrial; uterine sarcoma; vaginal cancer; childhood vaginal cancer; vascular tumors (soft tissue sarcoma); vulvar cancer; wilms tumor and other childhood kidney tumors; or cancer in young adults.

The methods, compositions, and systems of the present disclosure may be used to detect a cardiovascular disease state. As used herein, the terms “cardiovascular disease” (CVD) or “cardiovascular disorder” are used to classify numerous conditions affecting the heart, heart valves, and vasculature (e.g., veins and arteries) of the body and encompasses diseases and conditions including, but not limited to atherosclerosis, myocardial infarction, acute coronary syndrome, angina, congestive heart failure, aortic aneurysm, aortic dissection, iliac or femoral aneurysm, pulmonary embolism, atrial fibrillation, stroke, transient ischemic attack, systolic dysfunction, diastolic dysfunction, myocarditis, atrial tachycardia, ventricular fibrillation, endocarditis, peripheral vascular disease, and coronary artery disease (CAD). Further, the term cardiovascular disease refers to conditions in subjects that ultimately have a cardiovascular event or cardiovascular complication, referring to the manifestation of an adverse condition in a subject brought on by cardiovascular disease, such as sudden cardiac death or acute coronary syndrome, including, but not limited to, myocardial infarction, unstable angina, aneurysm, stroke, heart failure, non-fatal myocardial infarction, stroke, angina pectoris, transient ischemic attacks, aortic aneurysm, aortic dissection, cardiomyopathy, abnormal cardiac catheterization, abnormal cardiac imaging, stent or graft revascularization, risk of experiencing an abnormal stress test, risk of experiencing abnormal myocardial perfusion, and death.

As used herein, the ability to detect, diagnose or prognose cardiovascular disease, for example, atherosclerosis, can include determining if the patient is in a pre-stage of cardiovascular disease, has developed early, moderate or severe forms of cardiovascular disease, or has suffered one or more cardiovascular event or complication associated with cardiovascular disease.

Atherosclerosis (also known as arteriosclerotic vascular disease or ASVD) is a cardiovascular disease in which an artery-wall thickens as a result of invasion and accumulation and deposition of arterial plaques containing white blood cells on the innermost layer of the walls of arteries resulting in the narrowing and hardening of the arteries. The arterial plaque is an accumulation of macrophage cells or debris, and contains lipids (cholesterol and fatty acids), calcium and a variable amount of fibrous connective tissue. Diseases associated with atherosclerosis include, but are not limited to, atherothrombosis, coronary heart disease, deep venous thrombosis, carotid artery disease, angina pectoris, peripheral arterial disease, chronic kidney disease, acute coronary syndrome, vascular stenosis, myocardial infarction, aneurysm or stroke. In one embodiment the automated apparatuses, compositions, and methods of the present disclosure may distinguish the different stages of atherosclerosis, including, but not limited to, the different degrees of stenosis in a subject.

In some cases, the disease or disorder detected by the methods, compositions, or systems of the present disclosure is an endocrine disease. The term “endocrine disease” is used to refer to a disorder associated with dysregulation of endocrine system of a subject. Endocrine diseases may result from a gland producing too much or too little of an endocrine hormone causing a hormonal imbalance, or due to the development of lesions (such as nodules or tumors) in the endocrine system, which may or may not affect hormone levels. Suitable endocrine diseases able to be treated include, but are not limited to, e.g., Acromegaly, Addison's Disease, Adrenal Cancer, Adrenal Disorders, Anaplastic Thyroid Cancer, Cushing's Syndrome, De Quervain's Thyroiditis, Diabetes, Follicular Thyroid Cancer, Gestational Diabetes, Goiters, Graves' Disease, Growth Disorders, Growth Hormone Deficiency, Hashimoto's Thyroiditis, Hurthle Cell Thyroid Cancer, Hyperglycemia, Hyperparathyroidism, Hyperthyroidism, Hypoglycemia, Hypoparathyroidism,

Hypothyroidism, Low Testosterone, Medullary Thyroid Cancer, MEN 1, MEN 2A, MEN 2B, Menopause, Metabolic Syndrome, Obesity, Osteoporosis, Papillary Thyroid Cancer, Parathyroid Diseases, Pheochromocytoma, Pituitary Disorders, Pituitary Tumors, Polycystic Ovary Syndrome, Prediabetes, Silent, Thyroiditis, Thyroid Cancer, Thyroid Diseases, Thyroid Nodules, Thyroiditis, Turner Syndrome, Type 1 Diabetes, Type 2 Diabetes, and the like.

In some cases, the disease or disorder detected by methods, compositions, or systems of the present disclosure is an inflammatory disease. As referred to herein, inflammatory disease refers to a disease caused by uncontrolled inflammation in the body of a subject. Inflammation is a biological response of the subject to a harmful stimulus which may be external or internal such as pathogens, necrosed cells and tissues, irritants etc. However, when the inflammatory response becomes abnormal, it results in self-tissue injury and may lead to various diseases and disorders. Inflammatory diseases can include, but are not limited to, asthma, glomerulonephritis, inflammatory bowel disease, rheumatoid arthritis, hypersensitivities, pelvic inflammatory disease, autoimmune diseases, arthritis; necrotizing enterocolitis (NEC), gastroenteritis, pelvic inflammatory disease (PID), emphysema, pleurisy, pyelitis, pharyngitis, angina, acne vulgaris, urinary tract infection, appendicitis, bursitis, colitis, cystitis, dermatitis, phlebitis, rhinitis, tendonitis, tonsillitis, vasculitis, autoimmune diseases; celiac disease; chronic prostatitis, hypersensitivities, reperfusion injury; sarcoidosis, transplant rejection, vasculitis, interstitial cystitis, hay fever, periodontitis, atherosclerosis, psoriasis, ankylosing spondylitis, juvenile idiopathic arthritis, Behcet's disease, spondyloarthritis, uveitis, systemic lupus erythematosus, and cancer. For example, the arthritis includes rheumatoid arthritis, psoriatic arthritis, osteoarthritis or juvenile idiopathic arthritis, and the like.

The methods, compositions, and systems of the present disclosure may detect a neurological disease state. Neurological disorders or neurological diseases are used interchangeably and refer to diseases of the brain, spine and the nerves that connect them. Neurological diseases include, but are not limited to, brain tumors, epilepsy, Parkinson's disease, Alzheimer's disease, ALS, arteriovenous malformation, cerebrovascular disease, brain aneurysms, epilepsy, multiple sclerosis, Peripheral Neuropathy, Post-Herpetic Neuralgia, stroke, frontotemporal dementia, demyelinating disease (including but are not limited to, multiple sclerosis, Devic's disease (i.e. neuromyelitis optica), central pontine myelinolysis, progressive multifocal leukoencephalopathy, leukodystrophies, Guillain-Barre syndrome, progressing inflammatory neuropathy, Charcot-Marie-Tooth disease, chronic inflammatory demyelinating polyneuropathy, and anti-MAG peripheral neuropathy) and the like. Neurological disorders also include immune-mediated neurological disorders (IMNDs), which include diseases with at least one component of the immune system reacts against host proteins present in the central or peripheral nervous system and contributes to disease pathology. IMNDs may include, but are not limited to, demyelinating disease, paraneoplastic neurological syndromes, immune-mediated encephalomyelitis, immune-mediated autonomic neuropathy, myasthenia gravis, autoantibody-associated encephalopathy, and acute disseminated encephalomyelitis.

Methods, systems, and/or apparatuses of the present disclosure may be able to accurately distinguish between patients with or without Alzheimer's disease. These may also be able to detect patients who are pre-symptomatic and may develop Alzheimer's disease several years after the screening. This provides advantages of being able to treat a disease at a very early stage, even before development of the disease.

The methods, compositions, and systems of the present disclosure can detect a pre-disease stage of a disease or disorder. A pre-disease stage is a stage at which the patient has not developed any signs or symptoms of the disease. A pre-cancerous stage would be a stage in which cancer or tumor or cancerous cells have not be identified within the subject. A pre-neurological disease stage would be a stage in which a person has not developed one or more symptom of the neurological disease. The ability to diagnose a disease before one or more sign or symptom of the disease is present allows for close monitoring of the subject and the ability to treat the disease at a very early stage, increasing the prospect of being able to halt progression or reduce the severity of the disease.

The methods, compositions, and systems of the present disclosure may detect the early stages of a disease or disorder. Early stages of the disease can refer to when the first signs or symptoms of a disease may manifest within a subject. The early stage of a disease may be a stage at which there are no outward signs or symptoms. For example, in Alzheimer's disease an early stage may be a pre-Alzheimer's stage in which no symptoms are detected yet the patient will develop Alzheimer's months or years later.

Identifying a disease in either pre-disease development or in the early states may often lead to a higher likelihood for a positive outcome for the patient. For example, diagnosing cancer at an early stage (stage 0 or stage 1) can increase the likelihood of survival by over 80%. Stage 0 cancer can describe a cancer before it has begun to spread to nearby tissues. This stage of cancer is often highly curable, usually by removing the entire tumor with surgery. Stage 1 cancer may usually be a small cancer or tumor that has not grown deeply into nearby tissue and has not spread to lymph nodes or other parts of the body.

In some cases, the methods, compositions, and systems of the present disclosure are able to detect intermediate stages of the disease. Intermediate states of the disease describe stages of the disease that have passed the first signs and symptoms and the patient is experiencing one or more symptom of the disease. For example, for cancer, stage II or III cancers are considered intermediate stages, indicating larger cancers or tumors that have grown more deeply into nearby tissue. In some instances, stage II or III cancers may have also spread to lymph nodes but not to other parts of the body.

Further, the methods, compositions, and systems of the present disclosure may be able to detect late or advanced stages of the disease. Late or advanced stages of the disease may also be called “severe” or “advanced” and usually indicates that the subject is suffering from multiple symptoms and effects of the disease. For example, severe stage cancer includes stage IV, where the cancer has spread to other organs or parts of the body and is sometimes referred to as advanced or metastatic cancer.

The methods of the present disclosure can include processing the biomolecule corona data of a sample against a collection of biomolecule corona datasets representative of a plurality of diseases and/or a plurality of disease states to determine if the sample indicates a disease and/or disease state. For example, samples can be collected from a population of subjects over time. Once the subjects develop a disease or disorder, the present disclosure allows for the ability to characterize and detect the changes in biomolecule fingerprints over time in the subject by computationally analyzing the biomolecule fingerprint of the sample from the same subject before they have developed a disease to the biomolecule fingerprint of the subject after they have developed the disease. Samples can also be taken from cohorts of patients who all develop the same disease, allowing for analysis and characterization of the biomolecule fingerprints that are associated with the different stages of the disease for these patients (e.g. from pre-disease to disease states).

In some cases, the methods, compositions, and systems of the present disclosure are able to distinguish not only between different types of diseases, but also between the different stages of the disease (e.g. early stages of cancer). This can comprise distinguishing healthy subjects from pre-disease state subjects. The pre-disease state may be stage 0 or stage 1 cancer, a neurodegenerative disease, dementia, a coronary disease, a kidney disease, a cardiovascular disease (e.g., coronary artery disease), diabetes, or a liver disease. Distinguishing between different stages of the disease can comprise distinguishing between two stages of a cancer (e.g., stage 0 vs stage 1 or stage 1 vs stage 3).

### Computer Control Systems

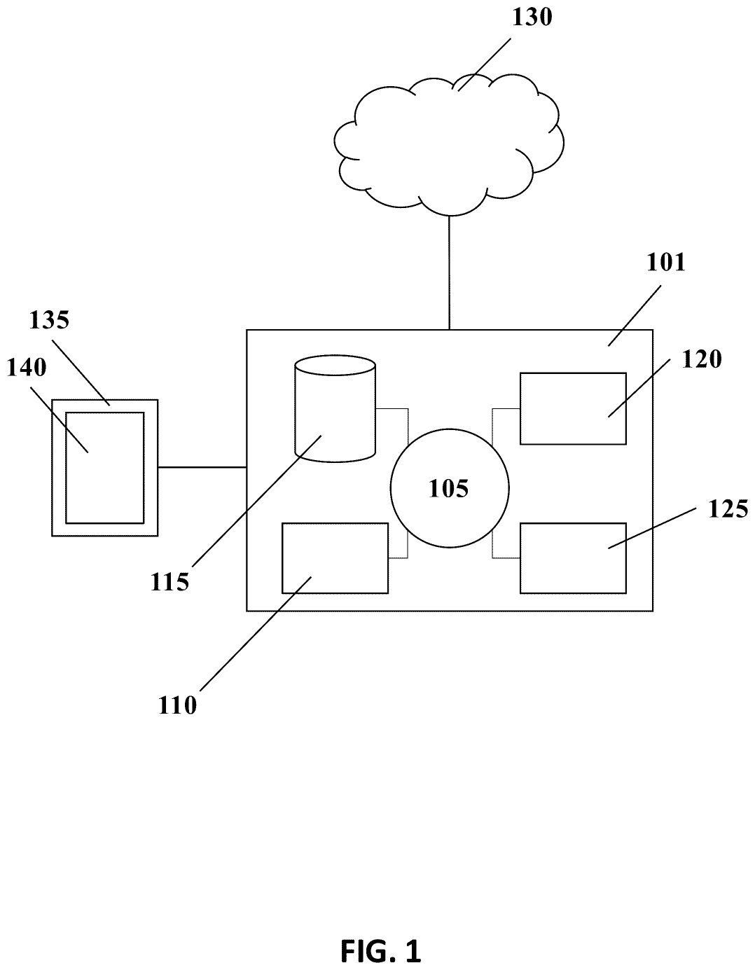

The present disclosure provides computer control systems that are programmed to implement methods of the disclosure. FIG. 1 shows a computer system that is programmed or otherwise configured to implement methods provided herein. The computer system 101 can regulate various aspects of the assays disclosed herein, which are capable of being automated (e.g., movement of any of the reagents disclosed herein on a substrate). The computer system 101 can be an electronic device of a user or a computer system that is remotely located with respect to the electronic device. The electronic device can be a mobile electronic device.

The computer system 101 includes a central processing unit (CPU, also “processor” and “computer processor” herein) 105, which can be a single core or multi core processor, or a plurality of processors for parallel processing. The computer system 101 also includes memory or memory location 110 (e.g., random-access memory, read-only memory, flash memory), electronic storage unit 115 (e.g., hard disk), communication interface 120 (e.g., network adapter) for communicating with one or more other systems, and peripheral devices 125, such as cache, other memory, data storage and/or electronic display adapters. The memory 110, storage unit 115, interface 120 and peripheral devices 125 are in communication with the CPU 105 through a communication bus (solid lines), such as a motherboard. The storage unit 115 can be a data storage unit (or data repository) for storing data. The computer system 101 can be operatively coupled to a computer network (“network”) 130 with the aid of the communication interface 120. The network 130 can be the Internet, an internet and/or extranet, or an intranet and/or extranet that is in communication with the Internet. The network 130 in some cases is a telecommunication and/or data network. The network 130 can include one or more computer servers, which can enable distributed computing, such as cloud computing. The network 130, in some cases with the aid of the computer system 101, can implement a peer-to-peer network, which may enable devices coupled to the computer system 101 to behave as a client or a server.

The CPU 105 can execute a sequence of machine-readable instructions, which can be embodied in a program or software. The instructions may be stored in a memory location, such as the memory 110. The instructions can be directed to the CPU 105, which can subsequently program or otherwise configure the CPU 105 to implement methods of the present disclosure. Examples of operations performed by the CPU 105 can include fetch, decode, execute, and writeback.

The CPU 105 can be part of a circuit, such as an integrated circuit. One or more other components of the system 101 can be included in the circuit. In some cases, the circuit is an application specific integrated circuit (ASIC).

The storage unit 115 can store files, such as drivers, libraries and saved programs. The storage unit 115 can store user data, e.g., user preferences and user programs. The computer system 101 in some cases can include one or more additional data storage units that are external to the computer system 101, such as located on a remote server that is in communication with the computer system 101 through an intranet or the Internet.

The computer system 101 can communicate with one or more remote computer systems through the network 130. For instance, the computer system 101 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 101 via the network 130.

Methods as described herein can be implemented by way of machine (e.g., computer processor) executable code stored on an electronic storage location of the computer system 101, such as, for example, on the memory 110 or electronic storage unit 115. The machine executable or machine readable code can be provided in the form of software. During use, the code can be executed by the processor 105. In some cases, the code can be retrieved from the storage unit 115 and stored on the memory 110 for ready access by the processor 105. In some situations, the electronic storage unit 115 can be precluded, and machine-executable instructions are stored on memory 110.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 101 can include or be in communication with an electronic display 135 that comprises a user interface (UI) 140 for providing, for example a readout of the proteins identified using the methods disclosed herein. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 105.

Determination, analysis or statistical classification is done by methods known in the art, including, but not limited to, for example, a wide variety of supervised and unsupervised data analysis and clustering approaches such as hierarchical cluster analysis (HCA), principal component analysis (PCA), Partial least squares Discriminant Analysis (PLSDA), machine learning (also known as random forest), logistic regression, decision trees, support vector machine (SVM), k-nearest neighbors, naive bayes, linear regression, polynomial regression, SVM for regression, K-means clustering, and hidden Markov models, among others. The computer system can perform various aspects of analyzing the protein sets or protein corona of the present disclosure, such as, for example, comparing/analyzing the biomolecule corona of several samples to determine with statistical significance what patterns are common between the individual biomolecule coronas to determine a protein set that is associated with the biological state. The computer system can be used to develop classifiers to detect and discriminate different protein sets or protein corona (e.g., characteristic of the composition of a protein corona). Data collected from the presently disclosed sensor array can be used to train a machine learning algorithm, specifically an algorithm that receives array measurements from a patient and outputs specific biomolecule corona compositions from each patient. Before training the algorithm, raw data from the array can be first denoised to reduce variability in individual variables.

Machine learning can be generalized as the ability of a learning machine to perform accurately on new, unseen examples/tasks after having experienced a learning data set. Machine learning may include the following concepts and methods. Supervised learning concepts may include AODE; Artificial neural network, such as Backpropagation, Autoencoders, Hopfield networks, Boltzmann machines, Restricted Boltzmann Machines, and Spiking neural networks; Bayesian statistics, such as Bayesian network and Bayesian knowledge base; Case-based reasoning; Gaussian process regression; Gene expression programming; Group method of data handling (GMDH); Inductive logic programming; Instance-based learning; Lazy learning; Learning

Automata; Learning Vector Quantization; Logistic Model Tree; Minimum message length (decision trees, decision graphs, etc.), such as Nearest Neighbor Algorithm and Analogical modeling; Probably approximately correct learning (PAC) learning; Ripple down rules, a knowledge acquisition methodology; Symbolic machine learning algorithms; Support vector machines; Random Forests; Ensembles of classifiers, such as Bootstrap aggregating (bagging) and Boosting (meta-algorithm); Ordinal classification; Information fuzzy networks (IFN); Conditional Random Field; ANOVA; Linear classifiers, such as Fisher's linear discriminant, Linear regression, Logistic regression, Multinomial logistic regression, Naive Bayes classifier, Perceptron, Support vector machines; Quadratic classifiers; k-nearest neighbor; Boosting; Decision trees, such as C4.5, Random forests, ID3, CART, SLIQ SPRINT; Bayesian networks, such as Naive Bayes; and Hidden Markov models. Unsupervised learning concepts may include; Expectation-maximization algorithm; Vector Quantization; Generative topographic map; Information bottleneck method; Artificial neural network, such as Self-organizing map; Association rule learning, such as, Apriori algorithm, Eclat algorithm, and FPgrowth algorithm; Hierarchical clustering, such as Singlelinkage clustering and Conceptual clustering; Cluster analysis, such as, K-means algorithm, Fuzzy clustering, DBSCAN, and OPTICS algorithm; and Outlier Detection, such as Local Outlier Factor. Semi-supervised learning concepts may include; Generative models; Low-density separation; Graph-based methods; and Co-training. Reinforcement learning concepts may include; Temporal difference learning; Q-learning; Learning Automata; and SARSA. Deep learning concepts may include; Deep belief networks; Deep Boltzmann machines; Deep Convolutional neural networks; Deep Recurrent neural networks; and Hierarchical temporal memory. A computer system may be adapted to implement a method described herein. The system includes a central computer server that is programmed to implement the methods described herein. The server includes a central processing unit (CPU, also “processor”) which can be a single core processor, a multi core processor, or plurality of processors for parallel processing. The server also includes memory (e.g., random access memory, read-only memory, flash memory); electronic storage unit (e.g. hard disk); communications interface (e.g., network adaptor) for communicating with one or more other systems; and peripheral devices which may include cache, other memory, data storage, and/or electronic display adaptors. The memory, storage unit, interface, and peripheral devices are in communication with the processor through a communications bus (solid lines), such as a motherboard. The storage unit can be a data storage unit for storing data. The server is operatively coupled to a computer network (“network”) with the aid of the communications interface. The network can be the Internet, an intranet and/or an extranet, an intranet and/or extranet that is in communication with the Internet, a telecommunication or data network. The network in some cases, with the aid of the server, can implement a peer-to-peer network, which may enable devices coupled to the server to behave as a client or a server.

The storage unit can store files, such as subject reports, and/or communications with the data about individuals, or any aspect of data associated with the present disclosure.

The computer server can communicate with one or more remote computer systems through the network. The one or more remote computer systems may be, for example, personal computers, laptops, tablets, telephones, Smart phones, or personal digital assistants.

In some applications the computer system includes a single server. In other situations, the system includes multiple servers in communication with one another through an intranet, extranet and/or the internet.

The server can be adapted to store measurement data or a database as provided herein, patient information from the subject, such as, for example, medical history, family history, demographic data and/or other clinical or personal information of potential relevance to a particular application. Such information can be stored on the storage unit or the server and such data can be transmitted through a network.

Methods as described herein can be implemented by way of machine (or computer processor) executable code (or software) stored on an electronic storage location of the server, such as, for example, on the memory, or electronic storage unit. During use, the code can be executed by the processor. In some cases, the code can be retrieved from the storage unit and stored on the memory for ready access by the processor. In some situations, the electronic storage unit can be precluded, and machine-executable instructions are stored on memory. Alternatively, the code can be executed on a second computer system.

Aspects of the systems and methods provided herein, such as the server, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless likes, optical links, or the like, also may be considered as media bearing the software. As used herein, unless restricted to non-transitory, tangible “storage” media, terms such as computer or machine “readable medium” can refer to any medium that participates in providing instructions to a processor for execution.

The computer systems described herein may comprise computer-executable code for performing any of the algorithms or algorithms-based methods described herein. In some applications the algorithms described herein will make use of a memory unit that is comprised of at least one database.

Data relating to the present disclosure can be transmitted over a network or connections for reception and/or review by a receiver. The receiver can be but is not limited to the subject to whom the report pertains; or to a caregiver thereof, e.g., a health care provider, manager, other health care professional, or other caretaker; a person or entity that performed and/or ordered the analysis. The receiver can also be a local or remote system for storing such reports (e.g. servers or other systems of a “cloud computing” architecture). In one embodiment, a computer-readable medium includes a medium suitable for transmission of a result of an analysis of a biological sample using the methods described herein.

Aspects of the systems and methods provided herein can be embodied in programming. Various aspects of the technology may be thought of as “products” or “articles of manufacture” typically in the form of machine (or processor) executable code and/or associated data that is carried on or embodied in a type of machine readable medium. Machine executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide nontransitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

### Classification of Protein Corona Using Machine Learning

The method of determining a set of proteins associated with the disease or disorder and/or disease state include the analysis of the corona of the at least two samples. This determination, analysis or statistical classification is done by methods known in the art, including, but not limited to, for example, a wide variety of supervised and unsupervised data analysis, machine learning, deep learning, and clustering approaches including hierarchical cluster analysis (HCA), principal component analysis (PCA), Partial least squares Discriminant Analysis (PLS-DA), random forest, logistic regression, decision trees, support vector machine (SVM), k-nearest neighbors, naive bayes, linear regression, polynomial regression, SVM for regression, K-means clustering, and hidden Markov models, among others. In other words, the proteins in the corona of each sample are compared/analyzed with each other to determine with statistical significance what patterns are common between the individual corona to determine a set of proteins that is associated with the disease or disorder or disease state.

Generally, machine learning algorithms are used to construct models that accurately assign class labels to examples based on the input features that describe the example. In some case it may be advantageous to employ machine learning and/or deep learning approaches for the methods described herein. For example, machine learning can be used to associate the protein corona with various disease states (e.g. no disease, precursor to a disease, having early or late stage of the disease, etc.). For example, in some cases, one or more machine learning algorithms are employed in connection with a method of the invention to analyze data detected and obtained by the protein corona and sets of proteins derived therefrom. For example, in one embodiment, machine learning can be coupled with the sensor array described herein to determine not only if a subject has a pre-stage of cancer, cancer or does not have or develop cancer, but also to distinguish the type of cancer.

In some cases, quantities of molecules can be processed using methods, including, but not limited to, for example, a wide variety of supervised and unsupervised data analysis, machine learning, deep learning, modeling and clustering approaches including hierarchical cluster analysis (HCA), principal component analysis (PCA), Partial least squares Discriminant Analysis (PLS-DA), random forest, logistic regression, decision trees, support vector machine (SVM), k-nearest neighbors, naive Bayes, linear regression, polynomial regression, SVM for regression, K-means clustering, and hidden Markov models, differential equation and stochastic differential equation models, among others. In some embodiments, the machine learning algorithm may be trained to learn a latent representation of quantities of molecules. In some embodiments, the machine learning algorithm may be supervised learning algorithm.

Input features to a machine learning algorithm may comprise various kinds of information. In some cases, an input feature may comprise a value that represents a physicochemical property of a surface used to assay a biomolecule. A physicochemical property of a particle may comprise various properties disclosed herein, which includes: charge, hydrophobicity, hydrophilicity, amphipathicity, coordinating, reaction class, surface free energy, various functional groups/modifications (e.g., sugar, polymer, amine, amide, epoxy, crosslinker, hydroxyl, aromatic, or phosphate groups). In some cases, an input feature may comprise a value that represents a parameter of a given measurement. A parameter may comprise incubation conditions including temperature, incubation time, pH, buffer type, and any variables in performing a measurement disclosed herein. In some embodiments, the input datasets may include a series of quantity measurements at different conditions, but without any data representing the relative differences between the conditions.

In some cases, a clustering algorithm can refer to a method of grouping samples in a dataset by some measure of similarity. In some cases, samples can be grouped in a set space, for example, element ‘a’ is in set ‘A’. In some cases, samples can be grouped in a continuous space, for example, element ‘a’ is a point in Euclidean space with distance ‘l’ away from the centroid of elements comprising cluster ‘A’. In some cases, samples can be grouped in a graph space, for example, element ‘a’ is highly connected to elements comprising cluster ‘A’. In some cases, clustering can refer to the principle of organizing a plurality of elements into groups in some mathematical space based on some measure of similarity.

In some cases, clustering can comprise grouping any number of molecules or quantities in a dataset by any quantitative measure of similarity. In some cases, clustering can comprise K-means clustering. In some cases, clustering can comprise hierarchical clustering. In some cases, clustering can comprise using random forest models. In some cases, clustering can comprise boosted tree models. In some cases, clustering can comprise using support vector machines. In some cases, clustering can comprise calculating one or more N−1 dimensional surfaces in N-dimensional space that partitions a dataset into clusters. In some cases, clustering can comprise distribution-based clustering. In some cases, clustering can comprise fitting a plurality of prior distributions over the data distributed in N-dimensional space. In some cases, clustering can comprise using density-based clustering. In some cases, clustering can comprise using fuzzy clustering. In some cases, clustering can comprise computing probability values of a data point belonging to a cluster. In some cases, clustering can comprise using constraints. In some cases, clustering can comprise using supervised learning. In some embodiments, clustering can comprise using unsupervised learning.

In some cases, clustering can comprise grouping molecules based on similarity. In some cases, clustering can comprise grouping molecules based on quantitative similarity. In some cases, clustering can comprise grouping molecules based on one or more features of each molecule. In some cases, clustering can comprise grouping molecules based on one or more labels of each molecule. In some cases, clustering can comprise grouping molecules based on Euclidean coordinates in a numerical representation of molecules. In some cases, clustering can comprise grouping molecules based on protein structural groups or functional groups (e.g., protein structures, substructures, or functional groups from protein databases such as Protein Data Bank or CATH Protein Structure Classification database). In some cases, a protein structural group or functional group may comprise protein primary structure, secondary structure, tertiary structure, or quaternary structure. In some cases, a protein structural group or functional group may be based at least partially on alpha helices, beta sheets, relative distribution of amino acids with different properties (e.g., aliphatic, aromatic, hydrophilic, acidic, basic, etc.), structural families (e.g., TIM barrel and beta barrel fold), protein domains (e.g., Death effector domain). In some cases, a protein structural group or functional group may be based at least partially on functional or spatial properties (e.g., functional groups-group of immune globulins, cytokines, cytoskeletal biomolecules, etc.).

Unless otherwise defined, all technical terms used herein have the same meaning as commonly understood by one of ordinary skill in the art to which this invention belongs. As used in this specification and the appended claims, the singular forms “a,” “an,” and “the” include plural references unless the context clearly dictates otherwise. Any reference to “or” herein is intended to encompass “and/or” unless otherwise stated.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” “less than or equal to,” or “at most” precedes the first numerical value in a series of two or more numerical values, the term “no more than,” “less than” or “less than or equal to,” or “at most” applies to each of the numerical values in that series of numerical values. For example, less than or equal to 3, 2, or 1 is equivalent to less than or equal to 3, less than or equal to 2, or less than or equal to 1.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### Embodiments

In one aspect, provided herein is a method for fractionating a biological sample comprising a plurality of biomolecules, the method comprising: a) contacting the biological sample with a plurality of particles, thereby adsorbing the plurality of biomolecules comprising at least 3 different protein classes to surfaces of the plurality of particles to form at least one biomolecule corona on the plurality of particle; b) separating at least one particle from the plurality of particles based on a physicochemical property of the particle, wherein the particle comprises a subset of the plurality of biomolecules of the biological sample adsorbed thereto; c) desorbing the subset of the plurality of biomolecules of the biological sample from the particle; and d) collecting the desorbed subset of the plurality of biomolecules, thereby fractionating the biological sample.

In some embodiments, the physicochemical property is selected from the group consisting of composition, size, surface charge, hydrophobicity, hydrophilicity, roughness, density surface functionalization, surface topography, surface curvature, porosity, core material, shell material, shape, and any combination thereof.

In some embodiments, the different protein classes comprise at least 10 proteins, 20 proteins, 40 proteins, 60 proteins, 80 proteins, 100 proteins, 150 proteins, 200 proteins, or 1000 proteins.

In some embodiments, the method comprises contacting the biological sample with a plurality of particles, thereby adsorbing the plurality of biomolecules comprising at least 4 different protein classes to surfaces of the plurality of particles.

In some embodiments, the method comprises contacting the biological sample with a plurality of particles, thereby adsorbing the plurality of biomolecules comprising at least 5 different protein classes to surfaces of the plurality of particles.

In some embodiments, the different protein classes comprise different functions.

In some embodiments, the different protein classes comprise different cellular or subcellular localizations.

In some embodiments, the different protein classes comprise different biological states.

In some embodiments, the different protein classes comprise different particular structural motifs.

In some embodiments, the different protein classes comprise different post-translational modifications.

In some embodiments, desorbing the subset of the plurality of biomolecules of the biological sample from the particle comprises treating the particle with an enzyme selected from the group consisting of trypsin, chymotrypsin, endoproteinase Glu C, endoproteinase Lys C, elastase, subtilisin, proteinase K, thrombin, factor X, endoproteinase Arg C, papain, endoproteinase AspN, thermolysin, pepsin, aspartyl protease, cathepsin D, zinc mealloprotease, glycoprotein endopeptidase, aminopeptidase, prenyl protease, caspase, kex2 endoprotease, or any combination thereof.

In another aspect, provided herein is a method for enriching biomolecules from a complex biological sample, the method comprising: a) contacting the complex biological sample with a particle comprising a cleavable or releasable coating, thereby forming a biomolecule corona on the particle; wherein the biomolecule corona comprises a plurality of biomolecules from the complex biological sample; b) digesting or releasing the coating of the particle, thereby releasing a subset of the plurality of biomolecules from the particle; and c) collecting at least the subset of biomolecules, thereby enriching the at least the subset of the plurality of biomolecules from the complex biological sample.

In Some Embodiments, the Cleavable or Releasable Coating Comprises a Nucleic Acid, a Lipid, a Protein, a Metal, a Resin, a Degradable Polymer, or any Combination Thereof.

In some embodiments, digesting the cleavable or releasable coating of the particle comprises incubating the particle with a chemical.

In some embodiments, the chemical comprises an acid.

In some embodiments, the chemical comprises an enzyme.

In some embodiments, releasing a coating of the particle comprises heating the particle, freezing the particle, grinding the particle, subjecting the particle to ultrasound, or any combination thereof.

In another aspect, provided herein is a method for fractionating a biological sample, comprising: a) contacting the biological sample with at least one particle to allow biomolecules from the biological sample to adsorb to the particle to form at least one biomolecule corona on the at least one particle; b) contacting the particle with a first solution, thereby desorbing a first subset of biomolecules from among the plurality of biomolecules from the particle into the first solution; c) collecting the first subset of biomolecules from the first solution; d) contacting the particle with a second solution, thereby desorbing a second subset of biomolecules from among the plurality of biomolecules from the particle into the second solution; and e) collecting the second subset of biomolecules from the second solution.

In some embodiments, the first solution is different from the second solution.

In some embodiments, the first solution comprises a higher concentration of an organic solvent than the second solution.

In some embodiments, the first solution comprises a lower concentration of an organic solvent than the second solution.

In some embodiments, the first subset of biomolecules is different from the second subset of biomolecules.

In some embodiments, contacting the particle with a second solution is performed after contacting the particle with a first solution.

In another aspect, provided herein is a method for fractionating a biological sample, comprising: a) contacting the biological sample with a particle to allow biomolecules from the biological sample to adsorb to a surface of the particle to form at least one biomolecule corona on the particle; b) contacting the particle with a fluid flow to desorb a first subset of the plurality of biomolecules adsorbed to the particle at a first time and a second subset of the plurality of biomolecules adsorbed to the particle at a second time; and c) collecting at least a portion of the first subset or the second subset of the plurality of biomolecules, thereby fractionating the biological sample.

In some embodiments, the method further comprises immobilizing the particle to a surface.

In some embodiments, the second time is at least 1 second apart, 2 seconds apart, 3 seconds apart, 4 seconds apart, 5 seconds apart, 5 seconds apart, 6 seconds apart, 7 seconds apart, 8 seconds apart, 9 seconds apart, 10 seconds apart, 20 seconds apart, 30 seconds apart, 45 seconds apart, 1 minute apart, 2 minutes apart, 3 minutes apart, 4 minutes apart, 5 minutes apart, 5 minutes apart, 6 minutes apart, 7 minutes apart, 8 minutes apart, 9 minutes apart, 10 minutes apart, 15 minutes apart, 20 minutes apart, 25 minutes apart, 30 minutes apart, 45 minutes apart, 60 minutes apart, 1.5 hours apart, 2 hours apart, 3 hours apart, 4 hours apart, 5 hours apart, 5 hours apart, 6 hours apart, 7 hours apart, 8 hours apart, 9 hours apart, 10 hours apart, 15 hours apart, 20 hours apart, 25 hours apart, 30 hours apart, 45 hours apart, or 60 hours apart from the first time.

In some embodiments, the first subset of biomolecules is different from the second subset of biomolecules.

In some embodiments, the method further comprises collecting at least a portion of the first subset and the second subset of the plurality of biomolecules.

In another aspect, provided herein is a method for purifying a biomolecule from a biological sample, the method comprising: a) expressing the biomolecule in a cell, wherein the biomolecule is coupled to a particle affinity tag comprising an affinity for a surface of a particle; and b) adsorbing the particle affinity tag to the particle, thereby coupling the biomolecule to the particle.

In some embodiments, the method further comprises dissociating the particle affinity tag from the biomolecule, thereby releasing the biomolecule from the surface of the particle.

In some embodiments, the method further comprises subjecting the particle to a chemical condition to lower the affinity of the particle affinity tag for the surface of the particle. In some embodiments, the particle affinity tag comprises an antibody.

In some embodiments, the cell is a bacteria cell, an insect cell, a plant cell, or a mammalian cell.

In another aspect, provided herein is a method for rapidly fractionating a biological sample comprising a plurality of biomolecules, the method comprising: a) separately contacting a plurality of portions of the biological sample to a plurality of particle panels, thereby forming a plurality of particle-sample mixtures; b) incubating the particle-sample mixtures to permit adsorption of biomolecules from the plurality of biomolecules to the surfaces of particles of the plurality of particle panels; and c) collecting at least a subset of the particle-adsorbed biomolecules, thereby generating a plurality of enriched samples, wherein the subset of the particle-adsorbed biomolecules comprises at least 5% of the mass of the plurality of biomolecules, and wherein the incubating and the collecting are performed in less than 90 minutes.

In some embodiments, a particle of the plurality of particles adsorbs an average of at least 100 Daltons (Da) biomolecules per nm² of its surface area.

In some embodiments, a particle of the plurality of particles adsorbs an average of at least 1 protein per 15 nm² of its surface area.

In another aspect, provided herein is a method for fractionating a biological sample comprising a plurality of biomolecules, the method comprising: a) contacting the biological sample with a plurality of particles, thereby forming a plurality of biomolecule coronas on the plurality of particles; b) separating the plurality of particles from the biological sample, thereby generating a depleted sample from the biological sample; and c) chromatographically purifying a biomolecule from the depleted sample to produce a purified sample, the purified sample having a higher purity of the biomolecule than a sample chromatographically purified directly from the biological sample.

In some embodiments, the purified sample is at least 10% more pure of the biomolecule than a sample chromatographically purified directly from the biological sample.

In another aspect, provided herein is a method for fractionating a biological sample, the method comprising: a) contacting the biological sample with a plurality of particles, wherein the plurality of particles adsorb a plurality of biomolecules from the biological sample to form biomolecule coronas on the plurality of particles; b) separating at least a subset of the plurality of particles based at least in part on their zeta potentials; c) collecting at least a subset of the biomolecules adsorbed to a particle from among the plurality of particles; and d) desorbing at least a subset of the biomolecules from the particle, thereby fractionating the biological sample.

In some embodiments, the method further comprises desorbing the subset of the biomolecules from the particle.

In some embodiments, the method further comprises subjecting the plurality of particles to an electric field.

In some embodiments, the adsorption results in a change in zeta potentials of the particles.

In some embodiments, the method further comprises subjecting the plurality of particles to capillary electrophoresis, gel electrophoresis (e.g., 1D or 2D SDS-PAGE or agarose gel), isoelectric focusing (IEF), zone electrophoresis (ZE) or isotachophoresis.

In some embodiments, the particle is at least one of a nanoparticle, a microparticle, a micelle, a liposome, an iron oxide particle, a graphene particle, a silica particle, a protein-based particle, a polystyrene particle, a silver particle, a gold particle, a metal particle, a quantum dot, a superparamagnetic particle, and any combination thereof.

In some embodiments, the particle is any one of the particles listed in Table 1.

In another aspect, provided herein is a method for enriching a biological sample, the method comprising: a) contacting the biological sample with a first particle under a first condition, thereby adsorbing a first plurality of biomolecules from the biological sample to a surface of the first particle to form at least one biomolecule corona on the first particle; b) collecting at least a subset of the first plurality of biomolecules, thereby generating a first enriched sample comprising the at least the subset of the first plurality of biomolecules; c) contacting the first enriched sample to a second particle under a second condition, thereby adsorbing at least a second plurality of biomolecules from the first enriched sample; d) collecting at least a subset of the second plurality of biomolecules, thereby generating a second enriched sample comprising the at least the subset of the second plurality of biomolecules.

In some embodiments, the first particle is different from the second particle.

In some embodiments, the first particle or the second particle comprises at least one of a nanoparticle, a microparticle, a micelle, a liposome, an iron oxide particle, a graphene particle, a silica particle, a protein-based particle, a polystyrene particle, a silver particle, a gold particle, a metal particle, a quantum dot, a superparamagnetic particle, and any combination thereof.

In some embodiments, the first particle or the second particle is any one of the particles listed in Table 1.

In one aspect, provided herein is a method for fractionating a biological sample comprising a plurality of biomolecules, the method comprising: a) contacting the biological sample with a plurality of particles, thereby adsorbing the plurality of biomolecules to surfaces of the plurality of particles to form at least one biomolecule corona on the plurality of particles; b) separating at least one particle comprising a biomolecule corona from the plurality of particles to form a first fraction, wherein separating of the first fraction is based on a physicochemical property of the first fraction, wherein the at least one particle comprising the biomolecule corona in the first fraction comprises a first subset of the plurality of biomolecules of the biological sample; c) desorbing the first subset of the plurality of biomolecules of the biological sample in the first portion; d) separating at least one particle comprising a biomolecule corona from the remaining plurality of particles in step (b) to form a second fraction, wherein separating of the second fraction is based on a physicochemical property of the second fraction, wherein the at least one particle comprising the biomolecule corona in the second fraction comprises a second subset of the plurality of biomolecules of the biological sample; e) desorbing the second subset of the plurality of biomolecules of the biological sample in the second portion; and f) collecting the desorbed first and second subsets of the plurality of biomolecules, thereby fractionating the biological sample.

In some embodiments, the plurality of particles comprises at least two subsets of particles, each subset of particles differing by at least one physicochemical property.

In some embodiments, the physicochemical property is selected from the group consisting of composition, size, surface charge, hydrophobicity, hydrophilicity, roughness, density surface functionalization, surface topography, surface curvature, porosity, core material, shell material, shape, zeta potential, and any combination thereof.

In some embodiments, the adsorption of the plurality of biomolecules to the surface of the plurality of particles results in a change of a physicochemical property.

In some embodiments, the adsorption results in a change in zeta potentials of the particles.

In some embodiments, the adsorption of biomolecules increases the zeta potentials of the particles.

In some embodiments, the adsorption of biomolecules decreases the zeta potentials of the particles.

In some embodiments, the method comprises contacting the biological sample with a plurality of particles, thereby adsorbing the plurality of biomolecules comprising at least 3, at least 4, at least 5, at least 10, at least 20, at least 35, at least 50, at least 70, or at least 90 different protein groups to surfaces of the plurality of particles.

In some embodiments, the method comprises contacting the biological sample with a plurality of particles, thereby adsorbing the plurality of biomolecules comprising at least 100, at least 150, at least 200, at least 250, at least 300, at least 400, at least 500, at least 750, or at least 900 different protein groups to surfaces of the plurality of particles.

In some embodiments, the method comprises contacting the biological sample with a plurality of particles, thereby adsorbing the plurality of biomolecules comprising at least 1000, 1500, at least 2000, at least 5000, at least 10000, at least 20000, at least 20000, at least 50000, or at least 100000 different protein groups to surfaces of the plurality of particles.

In some embodiments, desorbing the subset of the plurality of biomolecules of the biological sample from the particle comprises treating the particle with an enzyme selected from the group consisting of trypsin, chymotrypsin, endoproteinase Glu C, endoproteinase Lys C, elastase, subtilisin, proteinase K, thrombin, factor X, endoproteinase Arg C, papain, endoproteinase AspN, thermolysin, pepsin, aspartyl protease, cathepsin D, zinc mealloprotease, glycoprotein endopeptidase, aminopeptidase, prenyl protease, caspase, kex2 endoprotease, or any combination thereof.

In another aspect, provided herein is a method for fractionating one or more portions of a biological sample, comprising: a) contacting the one or more portions of a biological sample with one or more particles to allow a plurality of biomolecules from the one or more portions of a biological sample to adsorb to the one or more particle panels to form at least one biomolecule corona on the one or more particles; b) contacting the one or more particles with a first solution, thereby desorbing a first subset of biomolecules from among the plurality of biomolecules from the one or more particle panels into the first solution; c) collecting the first subset of biomolecules from the first solution; d) contacting the one or more particles with a second solution, thereby desorbing a second subset of biomolecules from among the plurality of biomolecules from the one or more particles into the second solution; and e) collecting the second subset of biomolecules from the second solution.

In some embodiments, the first solution is different from the second solution.

In some embodiments, the first solution comprises a higher concentration of an organic solvent than the second solution.

In some embodiments, the first solution comprises a lower concentration of an organic solvent than the second solution.

In some embodiments, the method comprises contacting the one or more particles with a third solution, thereby desorbing a third subset of biomolecules from among the plurality of biomolecules from the one or more particles into the third solution and collecting the third subset of biomolecules from the third solution.

In some embodiments, the first subset of biomolecules is different from the second subset of biomolecules and the third subset of biomolecules.

In some embodiments, contacting the one or more particles with a second solution is performed prior to contacting the one or more particles with a third solution and after contacting the one or more particles with a first solution.

In some embodiments, the third solution is different from the first or second solution.

In some embodiments, the one or more particles comprise at least two particles, and the at least two particles comprise a particle panel.

What is claimed is:

1. A method for fractionating a biological sample comprising a plurality of biomolecules, the method comprising:
a) contacting the biological sample with a plurality of particles, thereby adsorbing the plurality of biomolecules to surfaces of the plurality of particles to form at least one biomolecule corona on the plurality of particles;
b) separating at least one particle comprising a biomolecule corona from the plurality of particles to form a first fraction, wherein separating of the first fraction is based on a physicochemical property of the first fraction, wherein the at least one particle comprising the biomolecule corona in the first fraction comprises a first subset of the plurality of biomolecules of the biological sample;
c) desorbing the first subset of the plurality of biomolecules of the biological sample in the first fraction;
d) separating at least one particle comprising a biomolecule corona from the remaining plurality of particles in step (b) to form a second fraction, wherein separating of the second fraction is based on a physicochemical property of the second fraction, wherein the at least one particle comprising the biomolecule corona in the second fraction comprises a second subset of the plurality of biomolecules of the biological sample;
e) desorbing the second subset of the plurality of biomolecules of the biological sample in the second fraction; and
f) collecting the desorbed first and second subsets of the plurality of biomolecules, thereby fractionating the biological sample.

2. The method of claim 1, wherein the plurality of particles comprises at least two subsets of particles, each subset of particles differing by at least one physicochemical property.

3. The method of claim 2, wherein the physicochemical property is selected from the group consisting of composition, size, surface charge, hydrophobicity, hydrophilicity, roughness, density surface functionalization, surface topography, surface curvature, porosity, core material, shell material, shape, zeta potential, and any combination thereof.

4. The method of claim 3, wherein the adsorption of the plurality of biomolecules to the surface of the plurality of particles results in a change of a physicochemical property.

5. The method of claim 4, wherein the adsorption results in a change in zeta potentials of the particles.

6. The method of claim 2, wherein the adsorption of biomolecules increases the zeta potentials of the particles.

7. The method of claim 2, wherein the adsorption of biomolecules decreases the zeta potentials of the particles.

8. The method of claim 1, wherein contacting the biological sample with a plurality of particles, thereby adsorbing the plurality of biomolecules comprising at least 3, at least 4, at least 5, at least 10, at least 20, at least 35, at least 50, at least 70, or at least 90 different protein groups to surfaces of the plurality of particles.

9. The method of claim 1, wherein contacting the biological sample with a plurality of particles, thereby adsorbing the plurality of biomolecules comprising at least 100, at least 150, at least 200, at least 250, at least 300, at least 400, at least 500, at least 750, or at least 900 different protein groups to surfaces of the plurality of particles.

10. The method of claim 1, wherein contacting the biological sample with a plurality of particles, thereby adsorbing the plurality of biomolecules comprising at least 1000, 1500, at least 2000, at least 5000, at least 10000, at least 20000, at least 20000, at least 50000, or at least 100000 different protein groups to surfaces of the plurality of particles.

11. The method of claim 1, wherein desorbing the subset of the plurality of biomolecules of the biological sample from the particle comprises treating the particle with an enzyme selected from the group consisting of trypsin, chymotrypsin, endoproteinase Glu C, endoproteinase Lys C, elastase, subtilisin, proteinase K, thrombin, factor X, endoproteinase Arg C, papain, endoproteinase AspN, thermolysin, pepsin, aspartyl protease, cathepsin D, zinc mealloprotease, glycoprotein endopeptidase, aminopeptidase, prenyl protease, caspase, kex2 endoprotease, or any combination thereof.

12. A method for fractionating one or more portions of a biological sample, comprising:
a) contacting the one or more portions of a biological sample with one or more particles to allow a plurality of biomolecules from the one or more portions of a biological sample to adsorb to the one or more particle panels to form at least one biomolecule corona on the one or more particles;
b) contacting the one or more particles with a first solution, thereby desorbing a first subset of biomolecules from among the plurality of biomolecules from the one or more particle panels into the first solution;
c) collecting the first subset of biomolecules from the first solution;
d) contacting the one or more particles with a second solution, thereby desorbing a second subset of biomolecules from among the plurality of biomolecules from the one or more particles into the second solution; and
e) collecting the second subset of biomolecules from the second solution.

13. The method of claim 12, wherein the first solution is different from the second solution.

14. The method of claim 13, wherein the first solution comprises a higher concentration of an organic solvent than the second solution.

15. The method of claim 13, wherein the first solution comprises a lower concentration of an organic solvent than the second solution.

16. The method of claim 12, wherein the method comprises contacting the one or more particles with a third solution, thereby desorbing a third subset of biomolecules from among the plurality of biomolecules from the one or more particles into the third solution and collecting the third subset of biomolecules from the third solution.

17. The method of claim 16, wherein the first subset of biomolecules is different from the second subset of biomolecules and the third subset of biomolecules.

18. The method of claim 16, wherein contacting the one or more particles with a second solution is performed prior to contacting the one or more particles with a third solution and after contacting the one or more particles with a first solution.

19. The method of claim 16, wherein the third solution is different from the first or second solution.

20. The method of claim 16, wherein the one or more particles comprise at least two particles, and the at least two particles comprise a particle panel.
